(19) United States
(12) Patent Application Publication
(10) Pub. No.: US 2014/0045196 A1
(43) Pub. Date: Feb. 13, 2014

(54) METHODS OF PROGNOSIS AND DIAGNOSIS OF CANCER

(71) Applicants: University of Tokyo, Tokyo (JP); Abbott Japan Co. Ltd, Chiba-Ken (JP)

(72) Inventors: Noahiko Koshikawa, Tokyo (JP); Masatoshi Nakagawa, Saitama (JP); Eisaku Yoshida, Chiba (JP); Toru Yoshimura, Matsudo (JP); Motoharu Seiki, Tokyo (JP)

(73) Assignees: University of Tokyo, Tokyo (JP); Abbott Japan Co., Ltd, Chiba-Ken (JP)

(21) Appl. No.: 13/962,494

(22) Filed: Aug. 8, 2013

Related U.S. Application Data
(60) Provisional application No. 61/682,462, filed on Aug. 13, 2012.

(51) Int. Cl. G01N 33/574

(52) U.S. Cl. CPC G01N 33/57488
USPC 435/7.92

(57) ABSTRACT

Provided are methods of diagnosing cancer in a patient by detecting the presence and/or amount of a biomarker of cancer in a sample from the patient. The methods and biomarkers may be used to develop an accurate prognosis for a patient having cancer or suspected of having cancer, or to accurately diagnose a patient having, or suspected of having cancer. The methods and biomarkers may be used to identify and/or classify a patient as a candidate for a cancer therapy.

The subject application claims priority to U.S. Provisional Application No. 61/682,462 filed on Aug. 13, 2012, herein incorporated in its entirety by reference.

### TECHNICAL FIELD

The disclosure relates to methods and immunoassay platforms for determining a prognosis, diagnosis, or risk identification of cancer in a patient by detecting a biomarker in the patient as well as determining amounts thereof. The biomarkers may be used to identify a patient with cancer, identify a patient as a candidate for cancer therapy, to classify a patient's risk of developing cancer, or to classify a patient's cancer stage or risk of progression of cancer, as well as to determine a diagnosis, prognosis, or a treatment regimen.

### BACKGROUND

Cancer remains a significant cause of morbidity and mortality in adults in the developed world. In some instances, improvements in cancer treatments have been able to increase patient survival times from diagnosis to death. However, the overall success of a cancer treatment often depends on early detection of the disease, which allows for therapy to begin before primary tumor expansion and/or metastatic growth ensues. Accordingly, methods and assays that provide for an early and/or more accurate diagnosis of cancer are desirable because such methods and assays can allow for early therapeutic intervention and can improve patient outcomes (e.g., quality of life, survival expectation, etc.).

The laminins are a group of heterotrimeric proteins found in the basal lamina and form part of the basement membrane. These proteins are classified based on the three non-identical polypeptides that complex with each other to form the laminin structure. These three polypeptides are identified as the alpha (α) chain, beta (β) chain, and gamma (γ) chain, and each has several molecular species (e.g., α1-α5, β1-β3, and γ1-γ2). Laminin 5 (or LN5) is known to be present in the basal lamina and is abundant in the basement membrane located between epithelial cells and the connective tissue backing the epithelial cells. The structure of LN5 is unique among the known laminins in that it is the only laminin to have a structure that includes a gamma-2 (γ2) chain, which when complexed with the α3 chain and the β3 chain forms LN5. Physiologically, LN5 is known to be produced by epithelial cells and can promote cell adhesion, proliferation, differentiation, and/or migration. For example, when LN5 is secreted from the epithelial cells, it is susceptible to protease degradation (e.g., by membrane-type 1 matrix metalloproteinase-1 (MT1-MMP)). In some instances LN5 is processed toward the N-terminal end of the gamma-2 chain sequence to generate a fragment that has EGF-like activity, including the promoting of cell migration and invasion [Koshikawa, et al., J. Cell Biol., (2000) 148:615-624].

Existing methods for the detection of LN5, including its processed form(s), have focused on either histological methods (e.g., immunostaining of tissues), urinalysis, or detection of the proteolytic fragments (e.g., the N-terminal fragment), and do not provide a convenient and/or sensitive method for the detection of laminin gamma-2 monomer.

### SUMMARY OF THE INVENTION

In one aspect, the disclosure provides a method for providing a diagnosis, prognosis or risk classification to a subject who has cancer or who is at risk of having cancer, the method comprising the steps of obtaining a biological sample comprising blood from the subject; determining the concentration of laminin gamma-2 monomer in the biological sample from the subject; comparing the laminin gamma-2 monomer concentration from the sample to a reference laminin gamma-2 monomer concentration value, wherein a laminin gamma-2 monomer concentration in the sample greater than the reference laminin gamma-2 monomer concentration value identifies the subject as having cancer or as having an increased risk of developing cancer.

In an aspect, the disclosure relates to a method for providing a diagnosis, prognosis or risk classification to a subject who has cancer or who is at risk of having cancer, the method comprising the steps of obtaining a biological sample comprising blood from the subject; determining the concentration of laminin gamma-2 monomer in the biological sample from the subject; and providing the concentration of laminin gamma-2 monomer to identify the subject as having cancer or having an increased risk of developing cancer when compared to a reference laminin gamma-2 monomer concentration value.

In a further aspect the disclosure relates to a method for providing a diagnosis, prognosis or risk classification to a subject who has cancer or who is at risk of having cancer, the method comprising the steps of obtaining a biological sample comprising blood from the subject; determining the concentration of laminin gamma-2 monomer in the biological sample from the subject; comparing the laminin gamma-2 monomer concentration from the sample to a reference laminin gamma-2 monomer concentration value; and providing the comparison, wherein when the comparison comprises a laminin gamma-2 monomer concentration in the sample greater than the reference laminin gamma-2 monomer concentration value the comparison identifies the subject as having cancer or having an increased risk of developing cancer.

In another aspect the disclosure provides a method for detecting, diagnosing, or prognosing cancer in a subject comprising determining the concentration of laminin gamma-2 monomer in a sample comprising blood from the subject, wherein the concentration of laminin gamma-2 monomer is determined by contacting an antibody that specifically binds to laminin gamma-2 monomer with the sample and detecting antibody binding, and wherein cancer is detected, diagnosed, or prognosed in the subject when the concentration of laminin gamma-2 monomer in the sample from the subject is higher relative to a reference laminin gamma-2 monomer concentration.

In yet another aspect the disclosure provides a method for detecting, diagnosing, or prognosing cancer in a subject comprising determining the concentration of laminin gamma-2 monomer in a sample comprising blood from the subject, wherein the concentration of laminin gamma-2 monomer is determined by contacting an antibody that specifically binds to laminin gamma-2 monomer with the sample and detecting antibody binding; and comparing the concentration of laminin gamma-2 monomer in the from the subject to a reference laminin gamma-2 monomer concentration, wherein cancer is detected, diagnosed, or prognosed in the subject when the concentration of laminin gamma-2 monomer in the sample from the subject is higher relative to the reference laminin gamma-2 monomer concentration.

In embodiments, the method of the above aspects can further comprise detecting at least one additional biomarker of cancer in the sample. In embodiments of the method, providing a diagnosis can be providing a diagnosis of cancer such as, for example, bladder cancer or colorectal cancer. In other embodiments of the method, providing a prognosis can be determining cancer disease stage, or can be determining the likelihood or risk that the subject will develop cancer such as, for example, an aggressive or invasive form of bladder or colorectal cancer.

The method may further comprise the assessment of at least one additional biomarker of cancer selected from the group consisting of a laminin gamma-2 fragment (e.g., EGF-like fragment), carcinoembryonic antigen (CEA), carbohydrate antigen 19-9 (also called cancer antigen 19-9, or CA19-9) and the like. Assessment of the additional biomarker may comprise, for example, measuring the concentration of the biomarker in the biological sample from the subject, or may comprise a clinical evaluation of the subject. For an additional biomarker assessed by measuring the concentration of the biomarker in the biological sample from the subject, the method may further comprise comparing the measured concentration of the at least one additional biomarker with a reference value for the biomarker. The reference value for any of the additional biomarkers used in the methods disclosed herein can relate to the biomarker concentration of a control sample, a biomarker cutoff value, or a median concentration of a plurality of control samples from a group of control subjects, and the like.

In one aspect, the disclosure provides a method for identifying a subject as a candidate for a bladder cancer or colorectal cancer therapeutic regimen, the method comprising determining the concentration of laminin gamma-2 monomer in a biological sample comprising sera from the subject, and comparing the laminin gamma-2 monomer concentration in the sample to a reference laminin gamma-2 monomer concentration value, wherein when the laminin gamma-2 monomer concentration in the sample is greater than the reference laminin gamma-2 monomer concentration value, the subject is identified as a candidate for a cancer therapeutic regimen. In embodiments, the method can further comprising detecting at least one additional biomarker of cancer in the sample.

In another embodiment, the disclosure provides a method for the diagnosis, prognosis and/or risk classification of a subject having or at risk of having cancer such as, for example, bladder cancer or colorectal cancer, wherein the method comprises detecting an increased laminin gamma-2 monomer concentration in the subject relative to a control subject not having cancer.

In any of the methods disclosed herein, the laminin gamma-2 monomer reference value can be the laminin gamma-2 monomer concentration of a control sample or a laminin gamma-2 monomer cutoff value. The laminin gamma-2 monomer concentration can be, for example, the reference value for laminin gamma-2 monomer concentration in blood (e.g., plasma or serum). The control sample can be a biological sample of a control subject or a laminin gamma-2 monomer standard. The laminin gamma-2 monomer concentration of a control sample can be, for example, the median laminin gamma-2 monomer concentration of a plurality of control samples from a group of control subjects. Alternatively, a laminin gamma-2 monomer cutoff value can be determined by a receiver operating curve (ROC) analysis from biological samples of a patient group. Alternatively, a laminin gamma-2 monomer cutoff value can be determined by a quartile analysis of biological samples of a patient group. Still further alternatively, a laminin gamma-2 monomer cutoff value can be determined by a mean plus two standard deviation analysis of biological samples of a patient group. For example, a laminin gamma-2 monomer cutoff value can be determined by selecting a value that corresponds to the median of a patient group consisting of patients with cancer such as, for example, bladder cancer or colorectal cancer, which can be about 900-to about 1000 pg/ml serum. Alternatively, a laminin gamma-2 monomer cutoff value can be determined by selecting a value that corresponds to the 75^{th} percentile of a patient group consisting of patients having bladder cancer or colorectal cancer, which can be for example about 1,100 to about 1,400 pg/mL serum. In other embodiments, an appropriate cutoff value can be about 70 pg/mL to about 2,500 pg/mL in serum. For example, a cutoff value of about 1,000 pg/mL serum may be used to discriminate bladder cancer or colorectal cancer specimens and normal specimens. Similar cutoff values can be used for blood plasma and whole blood samples.

In any of the methods, the method can be performed via immunoassay. An example of an antibody that can be employed in such an immunoassay is monoclonal antibody 2H2.

In any of the methods, the subject can be a human subject and the biological sample of the subject and/or the control sample can be taken from a human subject. In any of the methods, the biological sample can be from a tissue or a bodily fluid including for example, any one of whole blood, plasma, or serum, or any cell culture suspension or fraction thereof. In some embodiments of the methods described herein, the sample is whole blood, plasma, or serum, suitably, plasma or serum. A coagulation inhibitor can be added to any peripheral blood sample. In the methods, determining the concentration of laminin gamma-2 monomer, and optionally the at least one additional biomarker, can be performed by an immunological assay method in which a reagent capable of specific binding to laminin gamma-2 monomer, and optionally a reagent capable of specific binding to the additional biomarker, are used.

In another aspect, the disclosure provides a kit for performing any of the methods and assays disclosed herein, wherein the kit includes at least one reagent capable of specifically binding laminin gamma-2 monomer, allowing for quantification of the laminin gamma-2 monomer concentration in a biological sample from a subject, and a reference standard indicating a reference laminin gamma-2 monomer concentration. In a kit for performing a method for providing a diagnosis, prognosis or risk classification of a subject having or at risk of having cancer (e.g., bladder cancer or colorectal cancer), the kit may further comprise at least one reagent capable of specifically binding at least one additional biomarker of cancer in the biological sample, allowing for quantification of the concentration of the at least one additional biomarker in the biological sample, and a reference standard indicating a reference concentration of the at least one additional biomarker of cancer in the biological sample. In any of the kits, the at least one reagent capable of specifically binding laminin gamma-2 monomer may comprise at least one antibody capable of specifically binding laminin gamma-2 monomer. In some embodiments, the kit is adapted for use with an ELISA assay.

### BRIEF DESCRIPTION OF THE DRAWINGS

FIG. 1 depicts serum concentration of laminin gamma 2 monomer (pg/mL) in various specimens (bladder, pancreas, ovarian, colon, stomach, esophagus cancers; and control samples). FIGS. 1A and 1B depict the same data, with 1B expanding the y-axis over the 0 pg/mL to 2000 pg/mL concentration range. Mean concentration (solid line) and one standard deviation (broken line) are indicated.

FIG. 2 is a Receiver Operating Characteristic (ROC) plot of the biomarkers carcinoembryonic antigen (CEA), carbohydrate antigen 19-9 (CA19-9), and laminin γ-2 monomer (laminin γ-2). FIG. 2A depicts the biomarkers in bladder cancer. FIG. 21 depicts the biomarkers in colorectal cancer.

FIG. 3 depicts the results of a Western blot analysis for the monoclonal antibodies D4B5 and 2H2 (each at 1 μg/mL). The data show that monoclonal antibody 2H2 specifically binds laminin gamma-2 monomer, and does not bind laminin gamma-2 monomer when it forms the laminin 5 complex.

FIG. 4 is a graphical representation of a data set from a dilution assay experiment using the 2H2 monoclonal antibody and various dilutions of laminin gamma-2 monomer (“g-2”) and assay matrix from a normal specimen (ABS001).

FIG. 5 illustrates a laminin gamma-2 monomer ELISA. FIG. 5A depicts a schematic cartoon of the general ELISA assay used in embodiments described herein. FIG. 5B depicts a laminin gamma-2 monomer ELISA standard curve between the concentration range of 0-4,000 pg/mL. The analytical sensitivity was determined to be 3.7 pg/mL.

FIG. 6 illustrates the analytical sensitivity of the ARCHITECT assay using sample diluent spiked with recombinant laminin gamma-2 monomer.

FIG. 7 illustrates the results of a further evaluation of the dilution linearity using normal speciments.

FIG. 8 illustrates the measurement of laminin gamma-2 monomer level in normal specimens.

### DETAILED DESCRIPTION

The disclosure is based on several unexpected developments and discoveries. In one general sense, the disclosure relates to the use of laminin gamma 2 monomer as a biomarker for the diagnosis, prognosis, and risk classification of certain cancers such as, for example, bladder and colorectal cancers. In another general sense, the disclosure relates to the surprising development of methods, assays, and kits that provide for the detection of laminin gamma 2 monomer in a biological sample that comprises blood (e.g., whole blood, plasma, or serum). Taken together, these unexpected finding provide for methods and assays that have significant advantages over existing methods and assays for the general measurement and quantification of laminin gamma-2 monomer, as well as fragments thereof. Accordingly, the disclosure identifies a novel association between increased (i.e., higher) laminin gamma 2 monomer serum levels in patients having certain cancers, including bladder cancer and colorectal cancer (e.g., a subject who has cancer, a subject with an increased risk of developing cancer, identifying a subject as a candidate for cancer therapy). As disclosed generally herein through the exemplification of several non-limiting embodiments, the presence of increased concentrations or levels of laminin gamma 2 monomer in a biological sample comprising blood can be associated with cancer (e.g., colorectal and/or bladder cancer). The association between increased blood levels of laminin gamma 2 monomer and cancer is robust, predictive of disease stage, disease onset, clinical progression, and/or disease severity in cancer. In contrast to existing methods and assays (e.g., that rely on measurement of laminin gamma-2 monomer based on patient urine output and/or the proteolytic processing of laminin gamma-2 monomer to its EGF-like fragment), embodiments of the methods and assays provided herein comprise simple and convenient steps that can be readily obtained from any subject. Assessment levels of laminin gamma 2 monomer in blood can therefore improve on current methods and assays that are used to diagnose cancer, provide prognosis of cancer treatment or cancer severity, and/or to stratify or identify patient risk of developing cancer, thereby significantly benefiting patients having or at risk of developing cancer. Further, combined use of laminin gamma 2 monomer and additional biomarkers can provide additional advantages.

Accordingly, the disclosure provides methods of providing a diagnosis, prognosis or risk classification/identification of a subject or group of subjects having or at risk of having cancer such as bladder cancer or colorectal cancer, using laminin gamma 2 monomer as a clinical biomarker. Also provided are methods of identification of a candidate subject or group of candidate subjects for a cancer therapeutic regimen, such as treatments for bladder or colorectal cancer, where the methods utilize laminin gamma 2 monomer as a biomarker. The disclosure also provides kits for performing the disclosed methods.

Section headings as used in this section and the entire disclosure herein are merely for organizational purposes and are not intended to be limiting.

### A. DEFINITIONS

As used herein, the singular forms “,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the terms “including” and “having,” as well as other forms of those terms, such as “includes,” “included”, “has,” and “have” are not limiting.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, blood, serum or plasma sample, in accordance with the methods described herein and other methods known in the art. Some components can be in solution or lyophilized for reconstitution for use in an assay.

“Control” as used herein when referring to a composition, can refer to a composition known to not contain an analyte of interest (“negative”), e.g., laminin gamma 2 monomer (laminin gamma 2 monomer or variants of laminin gamma 2 monomer, or combinations thereof); or to contain an analyte of interest (“positive control”), e.g., laminin gamma 2 monomer (such as human laminin gamma 2 monomer, variants of laminin gamma 2 monomer, or any combinations thereof). A positive control can comprise a known concentration of laminin gamma 2 monomer. “Control,” “positive control,” and “calibrator” may be used interchangeably herein to refer to a composition comprising a known concentration of laminin gamma 2 monomer. A “positive control” can be used to establish assay performance characteristics and is a useful indicator of the integrity of reagents (e.g., analytes). A “normal control” or “healthy control” may refer to a sample or specimen taken from a subject, or an actual subject who does not have cancer, or is not at risk of developing cancer.

As used herein, the term “laminin gamma-2 monomer,” “laminin-5 gamma-2 monomer,” “LN-5 gamma-2 monomer,” “gamma-2 monomer,” “gamma-2,” “g-2 monomer” or any of the preceding terms with the “γ” symbol in place of the word “gamma” or the letter “g” are all interchangeable and refer to one of the polypeptide chains that constitutes laminin-5 (also known as “kalinin” and “nicein” among other synonyms) and is identified as the gamma (γ) chain (as opposed to the alpha (α) and beta (β) chains), of the gamma-2 molecular species (contrasting from the gamma-1 species). In some embodiments laminin gamma-2 monomer can relate to any laminin gamma-2 monomer sequence, including an amino acid sequence (e.g., protein, polypeptide, peptide (precursor or mature), fusions, derivatives, variants, etc. or a nucleic acid sequence encoding such an amino acid sequence (e.g., DNA or RNA fragments, truncations, fusions, derivatives, SNPs, variants, etc). Laminin gamma-2 monomer can be from any organism and, in some embodiments, comprises an amino acid sequence from higher eukaryotes, including mammals. In some non-limiting embodiments a laminin gamma-2 monomer can be selected from any of human (including isoforms a and b, UniProtKB/Swiss-Prot: Q13753; RefSeq NP_{—}0055532), mouse (M. musculus, UniProt: E9Q7G3; RefSeq NP_{—}032511.3) rat (R. norvegicus, GenBank: NP_{—}001094110 (precursor protein); UniProtKB/TrEMBL: F1LRH4) and chicken (G. gallus, GenBank AAS92197; UniProtKB/TrEMBL Q6PVZ6 (partial sequences)), as well as fly and worm.

In some embodiments laminin gamma-2 monomer comprises human laminin-5 gamma-2 monomer (encoded by GenBank accession no. NM_{—}005562 (mRNA), or the amino acid sequence associated with UniProtKB accession no. Q13753). In humans, the gene for laminin gamma-2 monomer (or “LAMC2”) is located on the q arm of chromosome 1 (1q25.3). Human laminin gamma-2 monomer sequences can include the precursor protein sequence that includes a signal peptide (usually amino acids 1-21) that is cleaved off to generate the mature secreted protein (amino acids 22-1193). Laminin gamma-2 monomer can also encompass any fusion protein as well as any amino acid sequence variants. As note above, laminin gamma-2 monomer is unique to laminin 5 which is predominantly localized in basil lamina and basement membrane.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself may not be detectable but may become detectable upon reaction with yet another moiety. Use of the term “detectably labeled” is intended to encompass such labeling.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamrczyk et al., Bioorg. Med. Chem. Lett. 16: 1324-1328 (2006); Adarmezyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridiniurn compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 1: 714-724 (2000); Mattingly et al., In Luminescence Biotechnology Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use suitably includes an absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%, or at least about 40%, at least about 45%, at least about 50%, at least about 55%, at least about 60%, at least about 65%, at least about 70%, at least about 75%, at least about 80%, at least about 85%, at least about 90%, or at least about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Predetermined cutoff” and “predetermined level” refer generally to an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Pretreatment reagent,” e.g., lysis, precipitation and/or solubilization reagent, as used in a diagnostic or prognostic assay as described herein is one that lyses any cells and/or solubilizes any analyte that is/are present in a test sample. Pretreatment is not necessary for all samples, as described further herein. Among other things, solubilizing the analyte (e.g., laminin gamma-2 monomer) entails release (e.g., dissociation or decrease of binding) of the analyte from any endogenous binding proteins present in the sample such as a blood sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Sample,” “test sample,” “specimen,” “sample from a subject,” and “patient sample” may be used interchangeably herein. The sample, such as a sample of blood, tissue, urine, serum, plasma, amniotic fluid, cerebrospinal fluid, placental cells or tissue, endothelial cells, leukocytes, or monocytes, can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Series of calibrating compositions” refers to a plurality of compositions comprising a known concentration of laminin gamma-2 monomer, wherein each of the compositions differs from the other compositions in the series by the concentration of laminin gamma-2 monomer.

“Specific binding partner” refers to a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Tracer” as used herein refers to an analyte or analyte fragment conjugated to a label, such as laminin gamma-2 monomer conjugated to a fluorescein moiety, wherein the analyte conjugated to the label can effectively compete with the analyte for sites on an antibody specific for the analyte.

As used herein, the term “cancer” refers to any malignant disease associated with unregulated cell proliferation, growth, invasion, and metastasis, or mass (e.g., angiogenic, neoplastic, or tumorigenic cell growth). In some embodiments, cancer can comprise bladder cancer or colorectal cancer.

Typically, bladder cancers originates from the cells lining the bladder (called transitional cells), and are classified based on the way they tumors grow. Papillary tumors are wart-like in appearance and are attached to a stalk. Nonpapillary (sessile) tumors are much less common but are more invasive and are typically associated with a worse outcome. A number of risk factors are associated with an increased likelihood of developing bladder cancer, including smoking, exposure to chemicals, long-term bladder infection as well as the chemotherapy drug cyclophosphamide and radiation treatment.

Bladder cancer is usually associated with a number of symptoms that can include abdominal pain, blood in the urine, bone pain, fatigue, pain while urinating, frequent and/or urgent urination, incontinence, and weight loss. Existing tests for detecting bladder cancer include abdominal CT scan, pelvic CT scan, bladder biopsy, cystoscopy, intravenous pyelogram, urinalysis, and urine cytology. Bladder cancer is typically staged scale ranked between 0 and IV, where Stage 0 involves non-invasive tumors that are only in the bladder lining; Stage I involves penetration through the bladder lining, but does not reach the muscle layer of the bladder; Stage II involves the tumor reaching muscle layer; Stage III involves the tumor penetrating through the muscle into tissue surrounding the bladder; and Stage IV involves metastatic disease (e.g., to neighboring lymph nodes or remote sites). When bladder cancer spreads, it often is first seen in organs and tissues including the prostate, rectum, ureter, uterus, and vagina. Metastatic bladder cancer often involves bone, liver, and/or lungs.

Treatments are typically based on the various stages of the disease, wherein treatment for disease at Stages 0 and I include surgery to remove the tumor (e.g., local partial resection) with chemotherapy and/or immunotherapy directed specifically to the bladder. At stages II and III, therapy can involve the partial or complete removal of the bladder, followed by radiation and chemotherapy, pre-surgical chemotherapy to attempt to shrink tumors before surgery, or a combination of radiation and chemotherapy for patients not eligible for surgery. At stage IV, bladder cancer is typically regarded as terminal and the treatment course typically includes chemotherapy.

Patients with stage 0 or I bladder cancer have a fairly good prognosis. While there is a high risk that the cancer will return, most bladder cancers that return can be surgically removed and cured. The cure rates for people with stage III tumors are less than 50%. Patients with stage IV bladder cancer are rarely cured.

In some embodiments cancer can comprise colorectal (or colon) cancer, which typically is a carcinoma that originates in the large intestine (colon) or the rectum (end of the colon). In the United States, colorectal cancer is often cited as one of the leading causes of cancer-related deaths. Early diagnosis is often associated with a complete cure of the disease. While there is no single cause of colon cancer, almost all colon cancers originates as polyps that are initially benign and slowly develop into malignant cancer. Risk factors that are associated with colorectal cancer include age (older than 60), smoking, alcohol consumption, diets high in red and/or processed meat, colorectal polyps, inflammatory bowel disease (e.g., ulcerative colitis or Crohn's disease), family history of colorectal cancer, genetic predisposition including Lynch syndrome and familial adenomatous polyposis (FAP).

In many instances colorectal cancer can exhibit no symptoms. However, some cases also present abdominal pain and tenderness, blood in the stool, diarrhea, constipation, narrow stool, and unexplained weight loss. As noted above, early detection of colorectal cancer often leads to excellent prognosis (cure). Existing tests and screens for colorectal cancer include physical examination of the abdomen, fecal occult blood test (FOBT), colonoscopy, sigmoidoscopy, and blood tests for anemia and proper liver function. The various stages of colon cancer (0-IV) are typically characterized as follows: Stage 0, cancer on the innermost layer of the intestine; Stage I, cancer in several inner layers of the colon; Stage II, cancer has spread to the muscle wall of the colon; Stage III, cancer has spread to the lymph nodes; Stage IV, cancer has spread to other organs.

Treatment of colorectal cancer can include any one or combination of surgery (e.g., colectomy), chemotherapy, and radiation therapy, which is typically dependent the stage of the disease. Typically patients in whom colorectal cancer is detected and treated early (e.g., Stages 0-III) can survive 5 years post-diagnosis and be deemed cured of the disease. Stage IV colorectal cancer is typically considered curable.

A diagnosis of cancer is typically made by any one or more clinical or diagnostic tests as noted above, and can include any one or combination of physical examinations, imaging tests, radiographs (X-rays), and lab diagnostics as described herein or as known in the art. Several biomarkers including carcinoembryonic antigen (CEA) and carbohydrate antigen 19-9 (CA19-9) are used in the diagnosis of colorectal cancer.

CEA is a glycoprotein involved in cell adhesion and is normally produced during fetal development and halts before birth. CEA was first detected in tissue extracts from human colon cancer extracts and increased levels in serum has been associated with colorectal carcinoma, as well as carcinomas of the gastric organs, pancreas, lung, breast, and medullary thyroid. A normal level of CEA is about 2.5 ng/mL. Nevertheless, the CEA marker is not completely reliable for diagnosing cancer or as a screening test for early detection of cancer.

CA19-9, while associated and identified as a marker for colon and pancreatic cancers, has been associated with high occurrence of both false negative results, as well as false positive results. Further, in patients who lack the Lewis antigen CA19-9 is not expressed even when the patient is afflicted with a tumor. Nevertheless, because CA19-9 can be elevated in many types of gastrointestinal cancer, such as colorectal cancer, esophageal cancer and hepatocellular carcinoma it finds use as a cancer biomarker (e.g., colorectal cancer).

Thus, both CEA and CA 19-9 have been associated with certain types of cancer and are used as biomarkers to diagnose the disease (e.g., bladder cancer or colorectal cancer). However, these markers lack the desirable sensitivity and specificity that is needed for the accurate and/or early diagnosis of cancer in patients.

As used herein, the terms “risk assessment,” “risk classification,” “risk identification,” or “risk stratification” of subjects (e.g., patients) refers to the evaluation of factors including biomarkers, to predict the risk of occurrence of future events including disease onset or disease progression, so that treatment decisions regarding the subject may be made on a more informed basis.

As used herein, the term “cancer risk” or “risk of developing cancer” of a subject refers to the evaluation of factors including biomarkers, to predict the risk of occurrence of cancer including increased probability of cancer onset, cancer progression, and occurrence/severity of clinical symptoms associated with cancer. In addition to laminin gamma-2 monomer levels, other factors that may indicate a poor disease prognosis include tumor size, disease Stage, serum concentrations of CA19-9 and/or CEA, family and/or personal history of cancer, and increased clinical severity of any presenting symptoms. Thus, in some embodiments, the method relate to providing a prognosis of cancer onset or cancer progression comprising detecting/determining the level of laminin gamma-2 monomer in a sample from a patient, in combination with any one or more prognostic factors described herein, or known in the art.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present disclosure include, but are not limited to, antiserum, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. In some embodiments, the subject may be a human patient at risk for developing or already having cancer such as, for example, bladder cancer or colorectal cancer.

The terms “sample” and “biological sample” as used herein generally refer to a biological material being tested for and/or suspected of containing an analyte of interest such as laminin gamma-2 monomer. The sample may be any tissue sample taken or derived from the subject. In some embodiments, the sample from the subject may comprise protein. In some embodiments, the sample from the subject may comprise nucleic acid (e.g., polynucleotide, mRNA, etc.).

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing urine, blood, serum and plasma, and other body fluids, are used in the practice of the present disclosure, for instance, when the antibodies provided herein are employed as immunodiagnostic reagents, and/or in laminin gamma-2 monomer immunoassay kit. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

The pretreatment reagent can be any reagent appropriate for use with the assay, e.g., immunoassay, and kit described herein. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, European Pat. Pub. No. 0 471 293, and U.S. Pat. App. Pub. No. 2008/0020401 (incorporated by reference in its entirety for its teachings regarding pretreatment). The pretreatment reagent can be a heterogeneous agent or a homogeneous agent.

With use of a heterogeneous pretreatment reagent, the pretreatment reagent precipitates analyte binding protein (e.g., protein that can bind to laminin gamma-2 monomer) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner laminin gamma-2 monomer, or variants of laminin gamma-2 monomer, such as a labeled anti-laminin gamma-2 monomer monoclonal antibody (or an antigenically reactive fragment thereof). The pretreatment reagent employed for such an assay typically is diluted in the pretreated test sample mixture, either before or during capture by the first specific binding partner. Despite such dilution, a certain amount of the pretreatment reagent (for example, 5 M methanol and/or 0.6 M ethylene glycol) is still present (or remains) in the test sample mixture during capture.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHODS

The methods encompass providing a diagnosis or a prognosis of a subject which includes, with respect to cancer, any one or more of determining the that the subject has cancer, determining the severity of cancer, determining the subject's risk for developing cancer (i.e., likelihood of disease onset), determining the efficacy of a cancer treatment regimen, identifying a subject as a candidate for cancer therapy, and risk assessment regarding progression of cancer in a subject having the disease. The methods are based in part on the unexpected finding that laminin gamma-2 monomer concentration in a biological sample (e.g., blood, serum, or plasma) from a subject is predictive or diagnostic of cancer (e.g., bladder or colorectal cancer) in the subject, and thus laminin gamma-2 monomer can be used as a prognostic or diagnostic biomarker for cancer.

The methods involve providing or obtaining a biological sample from the subject, which can be obtained by any known means including needle stick, needle biopsy, swab, and the like. In an embodiment of the method, the biological sample is a blood sample, preferably a blood plasma or serum sample, which may be obtained by any standard technique such as, for example, by venipuncture. Biological samples used in the methods may be stored or banked under suitable tissue storage conditions, or can be accessed from samples that have been previously stored or banked under suitable conditions. In some embodiments, the methods comprise reviewing data from a prior assay or analysis of a biological sample from the subject (e.g., measurement of laminin gamma-2 monomer and/or another cancer biomarker such as, for example, any one or more of CEA and CA19-9).

The methods encompass a method for diagnosis, prognosis and/or risk stratification of cancer in a subject having or suspected of having cancer by determining laminin gamma-2 monomer concentration in the subject. Providing a diagnosis can be, for example, providing a diagnosis of cancer in a subject, where the subject can be previously undiagnosed as having with cancer, (or not identified as having a risk at risk of having cancer), suspected of having cancer, or not. Alternatively, or in addition, providing a prognosis can be, for example, determining cancer severity or Stage, or can be a risk assessment, i.e. determination of the likelihood that the subject will develop cancer. The methods also encompass identifying one or more patients or a subgroup of patients having an increased risk of developing cancer. A shared feature of all methods is the determination of concentration of laminin gamma-2 monomer in a biological sample as described herein, wherein an increased concentration of laminin gamma-2 monomer in the sample relative to a reference value for laminin gamma-2 monomer concentration is indicative of cancer, or increased risk of developing cancer.

The laminin gamma-2 monomer concentration is deemed increased in comparison to a reference value or predetermined level, i.e., the reference laminin gamma-2 monomer concentration value as described herein. For example, a laminin gamma-2 monomer serum concentration useful as a reference concentration value is about 500 pg/ml, but can be higher or lower, for example about 200 pg/ml or about 1000 pg/ml in serum (e.g., about 200, 250, 300, 350, 400, 450, 500, 550, 600, 650, 700, 750, 800, 850, 900, 950 or 1000 pg/ml or more). The laminin gamma-2 monomer concentration may be deemed increased as compared to the reference value when it is detectably higher (e.g., from about 1% to about 10% higher), or significantly higher, e.g. at least 20% higher (1.2 fold), at least 30% (1.3 fold) higher, at least 40% higher (1.4 fold), at least 50% higher (1.5 fold), at least 60% higher (1.6 fold), at least 70% higher (1.7 fold), at least 80% higher (1.8 fold), at least 100% higher (2.0 fold or double), at least 150% higher (2.5 fold), or at least 200% higher (3.0 fold or triple).

The presence, concentration or amount of laminin gamma-2 monomer in a biological sample may be readily determined using any suitable assay as is known in the art. Examples include, but are not limited to, immunoassay, such as sandwich immunoassay (e.g., monoclonal-polyclonal sandwich immunoassays, including radioisotope detection (radioimmnunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay, a capture reagent that specifically binds laminin gamma-2 monomer (or a portion thereof) of interest is attached to the surface of a mass spectrometry probe, such as a pre-activated protein chip array. The laminin gamma-2 monomer (variants of laminin gamma-2 monomer, or any combinations thereof) is then specifically captured on the biochip, and the captured laminin gamma-2 monomer is detected by mass spectrometry. Alternatively, the laminin gamma-2 monomer can be eluted from the capture reagent and detected by traditional MALDI (matrix-assisted laser desorption/ionization) or by SELDI. A chemiluminescent microparticle immunoassay, in particular one employing the ARCHITECT®, automated analyzer (Abbott Laboratories, Abbott Park, Ill.), is an example of a preferred immunoassay. Other methods include, for example, mass spectrometry and immunohistochemistry (e.g. with sections from tissue biopsies) using antibodies (monoclonal, polyclonal, chimeric, humanized, human, etc.) or fragments thereof that specifically bind laminin gamma-2 monomer. Anti-laminin gamma-2 monomer antibodies and fragments thereof can be produced according to methods known in the art as described herein. Alternatively, commercially available anti-laminin gamma-2 monomer antibodies can be used as described herein. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

Laminin gamma-2 monomer, or variants or any combinations thereof, may be analyzed using an immunoassay. The presence or amount of laminin gamma-2 monomer can be determined using antibodies and detecting specific binding to laminin gamma-2 monomer. If desired, one or more of the antibodies described herein can be used in combination with one or more commercially available monoclonal/polyclonal antibodies. Such antibodies are commercially available from companies such as LifeSpan Biosciences, Inc. (Seattle, Wash.), Acris Antibodies, Inc. (San Diego, Calif.), Raybiotech, Inc. (Norcross, Ga.), Atlas Antibodies (Stockholm, Sweden), Sigma-Aldrich (St. Louis, Mo.), IMGENEX (San Diego, Calif.), GeneTex (Irvine, Calif.), Abcam (Cambridge, Mass.), Novus Biologicals (Littleton, Colo.), Santa Cruz Biotechnology, Inc. (Santa Cruz, Calif.), Cell Sciences (Canton, Mass.), US Biological (Swampscott, Mass.), AbD Serotec (Raleigh, N.C.), R&D Systems, Inc. (Minneapolis, Minn.), Thermo Scientific Pierce Products (Rockford, Ill.), Abnova (Taiwan & Walnut, Calif.), and Enzo Life Sciences International, Inc. (Plymouth Meeting, Pa.).

Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA.

A heterogeneous format may be used. For example, after the test sample is obtained from a subject, a first mixture is prepared. The mixture contains the test sample being assessed for laminin gamma-2 monomer (including variants of laminin gamma-2 monomer or any combinations thereof) and a first specific binding partner, wherein the first specific binding partner and any laminin gamma-2 monomer contained in the test sample form a first specific binding partner-laminin gamma-2 monomer complex. Preferably, the first specific binding partner is an anti-laminin gamma-2 monomer antibody or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-laminin gamma-2 monomer complex is formed, any unbound laminin gamma-2 monomer is removed from the complex using any technique known in the art. For example, the unbound laminin gamma-2 monomer can be removed by washing. Suitably, however, the first specific binding partner is present in excess of any laminin gamma-2 monomer present in the test sample, such that all laminin gamma-2 monomer that is present in the test sample is bound by the first specific binding partner.

After any unbound laminin gamma-2 monomer is removed, a second specific binding partner is added to the mixture to form a first specific binding partner-laminin gamma-2 monomer-second specific binding partner complex. The second specific binding partner is preferably an anti-laminin gamma-2 monomer antibody that binds to an epitope on laminin gamma-2 monomer that differs from the epitope on laminin gamma-2 monomer bound by the first specific binding partner. Moreover, also preferably, the second specific binding partner is labeled with or contains a detectable label as described above.

As noted above, use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The Sandwich ELISA measures the amount of antigen between two layers of antibodies (i.e., a capture antibody (i.e., at least one capture antibody) and a detection antibody (i.e. at least one detection antibody). The capture antibody and the detection antibody bind to different epitopes on the antigen, e.g., laminin gamma-2 monomer. Desirably, binding of the capture antibody to an epitope does not interfere with binding of the detection antibody to an epitope. Either monoclonal or polyclonal antibodies may be used as the capture and detection antibodies in the sandwich E LISA.

Generally, at least two antibodies are employed to separate and quantify laminin gamma-2 monomer (inclusive of variants of laminin gamma-2 monomer or any combinations thereof) in a test sample. More specifically, the at least two antibodies bind to certain epitopes of laminin gamma-2 monomer or a portion of laminin gamma-2 monomer forming an immune complex which is referred to as a “sandwich”. One or more antibodies can be used to capture the laminin gamma-2 monomer (e.g., laminin gamma-2 monomer or variants of laminin gamma-2 monomer, or any combinations thereof) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. In other words, antibodies are selected so that the one or more first antibodies brought into contact with a test sample containing, or suspected of containing laminin gamma-2 monomer (e.g., laminin gamma-2 monomer or variants of laminin gamma-2 monomer, or any combinations thereof) do not bind to all or part of an epitope recognized by the second or subsequent antibodies, thereby not interfering with the ability of the one or more second detection antibodies to bind to the laminin gamma-2 monomer (e.g., laminin gamma-2 monomer, variants of laminin gamma-2 monomer, or any combinations thereof).

The antibodies may be used as a first antibody in said immunoassay. Preferably, the antibody immunospecifically binds to an epitope comprising at least three contiguous (3) amino acids of laminin gamma-2 monomer with a K_{D} of from 4.2×10⁻¹¹ M to 7.4×10⁻¹³ M. The immunoassay may comprise a second antibody that immunospecifically binds to an epitope comprising at least three contiguous (3) amino acids of laminin gamma-2 monomer, wherein the contiguous (3) amino acids to which the second antibody binds is different from the three (3) contiguous amino acids to which the first antibody binds. In some embodiments, the antibody can preferentially bind laminin gamma-2 monomer over laminin-5, or over a fragment of laminin gamma-2 monomer (e.g., the EGF-like fragment of laminin gamma-2 monomer).

In an embodiment, a test sample suspected of containing laminin gamma-2 monomer (e.g., laminin gamma-2 monomer, variants of laminin gamma-2 monomer, or any combinations thereof) can be contacted with at least one capture antibody (or antibodies) and at least one detection antibodies either simultaneously or sequentially. In the sandwich assay format, a test sample suspected of containing laminin gamma-2 monomer (e.g., laminin gamma-2 monomer, variants of laminin gamma-2 monomer, or any combinations thereof) is first brought into contact with the at least one capture antibody that specifically binds to a particular epitope under conditions which allow the formation of an antibody-laminin gamma-2 monomer complex. If more than one capture antibody is used, a multiple capture antibody-laminin gamma-2 monomer complex is formed. In a sandwich assay, the antibodies, preferably, the at least one capture antibody, are used in molar excess amounts of the maximum amount of laminin gamma-2 monomer or the laminin gamma-2 monomer variant expected in the test sample. For example, from about 5 μg/mL to about 1 mg/mL of antibody per mL of microparticle coating buffer may be used.

Optionally, prior to contacting the test sample with the at least one first capture antibody, the at least one capture antibody can be bound to a solid support which facilitates the separation the antibody-laminin gamma-2 monomer complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind laminin gamma-2 monomer or laminin gamma-2 monomer variant. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiinmide.

After the test sample suspected of containing laminin gamma-2 monomer (e.g., laminin gamma-2 monomer, variants of laminin gamma-2 monomer, or any combinations thereof) is brought into contact with the at least one capture antibody, the test sample is incubated in order to allow for the formation of a capture antibody (or capture antibodies)-laminin gamma-2 monomer complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the capture antibody (antibodies)-laminin gamma-2 monomer complex, the complex is then contacted with at least one detection antibody (under conditions which allow for the formation of a capture antibody (antibodies)-laminin gamma-2 monomer-detection antibody (antibodies) complex). If the capture antibody-laminin gamma-2 monomer complex is contacted with more than one detection antibody, then a capture antibody (antibodies)-laminin gamma-2 monomer-detection antibody (antibodies) detection complex is formed. As with the capture antibody, when the at least one detection (and subsequent) antibody is brought into contact with the capture antibody-laminin gamma-2 monomer complex, a period of incubation under conditions similar to those described above is required for the formation of the capture antibody (antibodies)-laminin gamma-2 monomer-detection antibody (antibodies) complex. Preferably, at least one detection antibody contains a detectable label. The detectable label can be bound to the at least one detection antibody prior to, simultaneously with or after the formation of the capture antibody (antibodies)-laminin gamma-2 monomer-detection antibody (antibodies) complex. Any detectable label known in the art can be used as discussed herein and known in the art.

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-laminin gamma-2 monomer complexes form. Alternatively, if a second specific binding partner is used and the second specific binding partner is detectably labeled with an acridinium compound, detectably labeled first specific binding partner-laminin gamma-2 monomer-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of laminin gamma-2 monomer or a variant thereof is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of laminin gamma-2 monomer (e.g., laminin gamma-2 monomer, variants of laminin gamma-2 monomer, or any combinations thereof) in the sample can be quantified. Specifically, the amount of laminin gamma-2 monomer in the sample is proportional to the intensity of the signal generated. The amount of laminin gamma-2 monomer present can be quantified by comparing the amount of light generated to a standard curve for laminin gamma-2 monomer or by comparison to a reference standard. The standard curve can be generated using serial dilutions or solutions of known concentrations of laminin gamma-2 monomer by mass spectroscopy, gravimetric methods, and other techniques known in the art.

In a chemiluminescent microparticle assay employing the ARCHITECT® (or its successor) analyzer, the conjugate diluent pH should be about 5.8+/−0.2, the microparticle coating buffer should be maintained at room temperature (i.e., at about 17 to about 27° C.), the microparticle coating buffer pH should be about 5.5+/−0.2, and the microparticle diluent pH should be about 6.0+/−0.2. Solids preferably are less than about 0.2%, such as less than about 0.15%, less than about 0.14%, less than about 013%, less than about 0.12%, less than about 0.11%, less than about 0.10%, less than about 0.09%, less than about 0.08%, less than about 0.07%, less than about 0.06%, less than about 0.05%, less than about 0.04%, or less than about 0.03%, such as about 0.025%.

In a forward competitive format, a aliquot of labeled laminin gamma-2 monomer (e.g., laminin gamma-2 monomer, variants of laminin gamma-2 monomer, or any combinations thereof) of a known concentration is used to compete with laminin gamma-2 monomer in a test sample for binding to laminin gamma-2 monomer antibody (such as an immobilized laminin gamma-2 monomer antibody).

In a forward competition assay, an immobilized antibody (such as a laminin gamma-2 monomer antibody) can either be sequentially or simultaneously contacted with the test sample and a labeled laminin gamma-2 monomer, or laminin gamma-2 monomer variant. The laminin gamma-2 monomer protein or laminin gamma-2 monomer variant can be labeled with any detectable label, including those detectable labels discussed above in connection with the anti-laminin gamma-2 monomer antibodies. In this assay, the antibody can be immobilized on to a solid support. Alternatively, the antibody can be coupled to an antibody, such as an anti-species antibody, that has been immobilized on a solid support, such as a microparticle.

The labeled laminin gamma-2 monomer (e.g., laminin gamma-2 monomer, variants of laminin gamma-2 monomer, or any combinations thereof), the test sample, and the antibody are incubated under conditions similar to those described above in connection with the sandwich assay format. Two different species of antibody-laminin gamma-2 monomer complexes may then be generated. Specifically, one of the antibody-laminin gamma-2 monomer complexes generated contains a detectable label while the other antibody-laminin gamma-2 monomer complex does not contain a detectable label. The antibody-laminin gamma-2 monomer complex can be, but does not have to be, separated from the remainder of the test sample prior to quantification of the detectable label. Regardless of whether the antibody-laminin gamma-2 monomer complex is separated from the remainder of the test sample, the amount of detectable label in the antibody-laminin gamma-2 monomer complex is then quantified. The concentration of laminin gamma-2 monomer (e.g., laminin gamma-2 monomer, variants of laminin gamma-2 monomer, or any combinations thereof) in the test sample can then be determined by comparing the quantity of detectable label in the antibody-laminin gamma-2 monomer complex to a standard curve. The standard curve can be generated using serial dilutions of laminin gamma-2 monomer (e.g., laminin gamma-2 monomer, variants of laminin gamma-2 monomer, or any combinations thereof) of known concentration, by mass spectroscopy, gravimetrically and by other techniques known in the art.

The antibody-laminin gamma-2 monomer complex can be separated from the test sample by binding the antibody to a solid support, such as the solid supports discussed above in connection with the sandwich assay format, and then removing the remainder of the test sample from contact with the solid support.

In a reverse competition assay, an immobilized laminin gamma-2 monomer (e.g., laminin gamma-2 monomer, variants of laminin gamma-2 monomer, or any combinations thereof) can either be sequentially or simultaneously contacted with a test sample and at least one labeled antibody. Preferably, the antibody specifically binds to an epitope comprising at least 3, at least 4, at least 5, at least 6, at least 7, at least 8, at least 9, or at least 10, at least 15, at least 20, at least 25 or at least 30 amino acids of laminin gamma-2 monomer.

The laminin gamma-2 monomer (e.g., laminin gamma-2 monomer, variants of laminin gamma-2 monomer, or any combinations thereof) can be bound to a solid support, such as the solid supports discussed above in connection with the sandwich assay format.

The immobilized laminin gamma-2 monomer (e.g., laminin gamma-2 monomer, variants of laminin gamma-2 monomer, or any combinations thereof), test sample, and at least one labeled antibody are incubated under conditions similar to those described above in connection with the sandwich assay format. Two different species of laminin gamma-2 monomer-antibody complexes are then generated. Specifically, one of the laminin gamma-2 monomer-antibody complexes generated is immobilized and contains a detectable label while the other laminin gamma-2 monomer-antibody complex is not immobilized and contains a detectable label. The non-immobilized laminin gamma-2 monomer-antibody complex and the remainder of the test sample are removed from the presence of the immobilized laminin gamma-2 monomer-antibody complex through techniques known in the art, such as washing. Once the non-immobilized laminin gamma-2 monomer antibody complex is removed, the amount of detectable label in the immobilized laminin gamma-2 monomer-antibody complex is then quantified. The concentration of laminin gamma-2 monomer (e.g., laminin gamma-2 monomer, variants of laminin gamma-2 monomer, or any combinations thereof) in the test sample can then be determined by comparing the quantity of detectable label in the laminin gamma-2 monomer-complex to a standard curve. The standard curve can be generated using serial dilutions of laminin gamma-2 monomer or laminin gamma-2 monomer variant of known concentration, by mass spectroscopy, gravimetrically and by other techniques known in the art.

In a fluorescence polarization assay, an antibody or functionally active fragment thereof may be first contacted with an unlabeled test sample suspected of containing laminin gamma-2 monomer (e.g., laminin gamma-2 monomer, variants of laminin gamma-2 monomer, or any combinations thereof) to form an unlabeled laminin gamma-2 monomer-antibody complex. The unlabeled laminin gamma-2 monomer-antibody complex is then contacted with a fluorescently labeled laminin gamma-2 monomer (e.g., laminin gamma-2 monomer, variants of laminin gamma-2 monomer, or any combinations thereof). The labeled laminin gamma-2 monomer competes with any unlabeled laminin gamma-2 monomer in the test sample for binding to the antibody or functionally active fragment thereof. The amount of labeled laminin gamma-2 monomer-antibody complex formed is determined and the amount of laminin gamma-2 monomer (e.g., laminin gamma-2 monomer, variants of laminin gamma-2 monomer, or any combinations thereof) in the test sample determined via use of a standard curve.

The antibody used in a fluorescence polarization assay specifically binds to an epitope comprising at least 3, at least 4, at least 5, at least 6, at least 7, at least 8, at least 9, at least 10, at least 15, at least 20, at least 25 or at least 30 amino acids of laminin gamma-2 monomer.

The antibody, labeled laminin gamma-2 monomer (e.g., laminin gamma-2 monomer, variants of laminin gamma-2 monomer, or any combinations thereof), test sample and at least one labeled antibody may be incubated under conditions similar to those described above in connection with the sandwich immunoassay.

Alternatively, an antibody or functionally active fragment thereof may be simultaneously contacted with a fluorescently labeled laminin gamma-2 monomer (e.g., laminin gamma-2 monomer, variants of laminin gamma-2 monomer, or any combinations thereof) and an unlabeled test sample suspected of containing laminin gamma-2 monomer (e.g., laminin gamma-2 monomer, variants of laminin gamma-2 monomer, or any combinations thereof) to form both labeled laminin gamma-2 monomer-antibody complexes and unlabeled laminin gamma-2 monomer-antibody complexes. The amount of labeled laminin gamma-2 monomer-antibody complex formed is determined and the amount of laminin gamma-2 monomer in the test sample determined via use of a standard curve. The antibody used in this immunoassay specifically may bind to an epitope comprising at least 3, at least 4, at least 5, at least 6, at least 7, at least 8, at least 9, at least 10, at least 15, at least 20, at least 25 or at least 30 amino acids of laminin gamma-2 monomer.

Alternatively, an antibody or functionally active fragment thereof is first contacted with a fluorescently labeled laminin gamma-2 monomer (e.g., laminin gamma-2 monomer, variants of laminin gamma-2 monomer, or any combinations thereof) to form a labeled laminin gamma-2 monomer-antibody complex. The labeled laminin gamma-2 monomer-antibody complex is then contacted with an unlabeled test sample suspected of containing laminin gamma-2 monomer (e.g., laminin gamma-2 monomer, variants of laminin gamma-2 monomer, or any combinations thereof). Any unlabeled laminin gamma-2 monomer in the test sample competes with the laminin gamma-2 monomer (e.g., laminin gamma-2 monomer, variants of laminin gamma-2 monomer, or any combinations thereof) for binding to the antibody or functionally active fragment thereof. The amount of labeled laminin gamma-2 monomer-antibody complex formed is used to determine the amount of laminin gamma-2 monomer in the test sample via use of a standard curve. The antibody used in this immunoassay specifically binds to an epitope comprising at least three 3, at least 4, at least 5, at least 6, at least 7, at least 8, at least 9, at least 10, at least 15, at least 20, at least 25, or at least 30 amino acids of laminin gamma-2 monomer.

Mass spectrometry (MS) analysis may be used alone or in combination with other methods. Other methods include immunoassays and those described above to detect specific polynucleotides. The mass spectrometry method may be used to determine the presence and/or quantity of one or more biomarkers. MS analysis may comprise matrix-assisted laser desorption/ionization (MALDI) time-of-flight (TOF) MS analysis, such as, for example, directed-spot MALDI-TOF or liquid chromatography MALDI-TOF analysis. In some embodiments, the MS analysis comprises electrospray ionization (ESI) MS, such as liquid chromatography (LC) ESI-MS. Mass analysis can be accomplished using commercially available spectrometers. Methods for utilizing MS analysis, including MALDI-TOF MS and ESI-MS, to detect the presence and quantity of biomarker peptides in biological samples may be used. See, for example, U.S. Pat. Nos. 6,925,389; 6,989,100; and 6,890,763 for guidance, each of which is incorporated herein by reference.

It may be desirable to include a control sample or a calibrator, such as a series of calibrators. The control sample may be analyzed concurrently with the sample from the subject as described above. The results obtained from the subject sample can be compared to the results obtained from the control sample. Standard curves may be provided, with which assay results for the biological sample may be compared. Such standard curves present levels as a function of assay units, i.e., fluorescent signal intensity, if a fluorescent label is used. Using samples taken from multiple donors, standard curves can be provided for control levels of the laminin gamma-2 monomer in normal tissue, as well as for “at-risk” levels of the laminin gamma-2 monomer in tissue taken from donors, who may have one or more of the characteristics set forth above.

Thus, in view of the above, a method of determining the presence, amount or concentration of laminin gamma-2 monomer (e.g., laminin gamma-2 monomer, variants of laminin gamma-2 monomer, or any combinations thereof) in a test sample is provided. The method comprises assaying the test sample for laminin gamma-2 monomer (e.g., laminin gamma-2 monomer, variants of laminin gamma-2 monomer, or any combinations thereof) by an immunoassay, for example, employing at least one antibody and at least one detectable label and comprising comparing a signal generated by the detectable label as a direct or indirect indication of the presence, amount or concentration of laminin gamma-2 monomer in the test sample to a signal generated as a direct or indirect indication of the presence, amount or concentration of laminin gamma-2 monomer in a calibrator. The calibrator is optionally, and is preferably, part of a series of calibrators in which each of the calibrators differs from the other calibrators in the series by the concentration of laminin gamma-2 monomer. One of the at least one antibody is an isolated antibody, which specifically binds to laminin gamma-2 monomer (e.g., laminin gamma-2 monomer, variants of laminin gamma-2 monomer, or any combinations thereof), wherein the antibody has a domain or region selected from (i) a variably heavy domain region, or (ii) a variably heavy domain region and a variable light domain region. Alternatively, one of the at least one antibody is an isolated antibody, which specifically binds to laminin gamma-2 monomer (e.g., laminin gamma-2 monomer, variants of laminin gamma-2 monomer, or any combinations thereof), wherein the antibody has (i) a variable heavy chain comprising a complementarity determining region (CDR)1, a CDR2, and a CDR3 and a variable light chain comprising a CDR1, a CDR2, and a CDR3. An example of at least one antibody that can be used are antibodies which specifically bind to laminin gamma-2 monomer such as those commercially available from companies such as LifeSpan Biosciences, Inc. (Seattle, Wash.), Acris Antibodies, Inc. (San Diego, Calif.), Raybiotech, Inc. (Norcross, Ga.), Atlas Antibodies (Stockholm, Sweden), Sigma-Aldrich (St. Louis, Mo.), IMGENEX (San Diego, Calif.), GeneTex (Irvine, Calif.), Abcam (Cambridge, Mass.), Novas Biologicals (Littleton, Colo.), Santa Cruz Biotechnology, Inc. (Santa Cruz, Calif.), Cell Sciences (Canton, Mass.), US Biological (Swampscott, Mass.), AbD Serotec (Raleigh, N.C.), R&D Systems, Inc. (Minneapolis, Minn.), Thermo Scientific Pierce Products (Rockford, Ill.), Abnova (Taiwan & Walnut, Calif.), and Enzo Life Sciences International, Inc. (Plymouth Meeting, Pa.).

The method can comprise (i) contacting the test sample with at least one capture antibody, which binds to an epitope on laminin gamma-2 monomer (e.g., laminin gamma-2 monomer, variants of laminin gamma-2 monomer, or any combinations thereof), so as to form a capture antibody/laminin gamma-2 monomer complex, (ii) contacting the capture antibody/laminin gamma-2 monomer complex with at least one detection antibody, which comprises a detectable label and binds to an epitope on laminin gamma-2 monomer (e.g., laminin gamma-2 monomer, variants of laminin gamma-2 monomer, or any combinations thereof) that is not bound by the capture antibody, to form a capture antibody/laminin gamma-2 monomer/detection antibody complex, and (iii) determining the amount of laminin gamma-2 monomer in the test sample based on the signal generated by the detectable label in the capture antibody/laminin gamma-2 monomer/detection antibody complex formed in (ii).

Alternatively, the method can comprise (i) contacting the test sample with at least one capture antibody, which binds to an epitope of laminin gamma-2 monomer (e.g., laminin gamma-2 monomer, variants of laminin gamma-2 monomer, or any combinations thereof) so as to form a capture antibody/laminin gamma-2 monomer complex, and simultaneously or sequentially, in either order, contacting the test sample with detectably labeled laminin gamma-2 monomer, which can compete with any laminin gamma-2 monomer (e.g., laminin gamma-2 monomer, variants of laminin gamma-2 monomer, or any combinations thereof) in the test sample for binding to the at least one capture antibody. Any laminin gamma-2 monomer (e.g., laminin gamma-2 monomer, variants of laminin gamma-2 monomer, or any combinations thereof) present in the test sample and the detectably labeled laminin gamma-2 monomer compete with each other to form a capture antibody/laminin gamma-2 monomer complex and a capture antibody/detectably labeled laminin gamma-2 monomer complex, respectively. The method further comprises (ii) determining the presence, amount or concentration of laminin gamma-2 monomer in the test sample based on the signal generated by the detectable label in the capture antibody/detectably labeled laminin gamma-2 monomer complex formed in (ii). The signal generated by the detectable label in the capture antibody/detectably labeled laminin gamma-2 monomer complex is inversely proportional to the amount or concentration of laminin gamma-2 monomer in the test sample.

In some embodiments, the methods can comprise any techniques and assays that are used in the art to measure the amount of laminin gamma-2 monomer in a sample. For example, a polyclonal, monoclonal, chimeric, humanized or human anti-laminin gamma-2 monomer antibody (Ab) can be attached directly or indirectly, e.g., via a sheep (or other species) anti-human Ab, to a solid support. Any laminin gamma-2 monomer, which is present in a sample and brought into contact with the solid support, is bound by the polyclonal, monoclonal, chimeric humanized or human anti-laminin gamma-2 monomer Ab. A biotin-labeled mouse anti-laminin gamma-2 monomer Ab also binds to the laminin gamma-2 monomer. Streptavidin, which is linked to horseradish peroxidase (HRPO), binds to the biotin on the mouse anti-laminin gamma-2 monomer Ab. Upon being contacted with o-phenylenediamine, the HRPO converts the o-phenylenediamine to 2,3-diaminophenazine, which is orange-brown in color and can be measured spectrophotometrically at 492 nm.

The method can further comprise diagnosing, determining a prognosis, or assessing the efficacy of a treatment (therapeutic or prophylactic) of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy. The method can be adapted for use in an automated system or a semi-automated system.

Generally, a predetermined level can be employed as a benchmark against which to assess results obtained upon assaying a test sample for laminin gamma-2 monomer (e.g., laminin gamma-2 monomer, variants of laminin gamma-2 monomer, or any combinations thereof). Generally, in making such a comparison, the predetermined level is obtained by running a particular assay a sufficient number of times and under appropriate conditions such that a linkage or association of analyte presence, amount or concentration with a particular stage or endpoint of a disease, disorder or condition (e.g. cancer) or with particular indicia can be made. Typically, the predetermined level is obtained with assays of reference subjects (or populations of subjects). The laminin gamma-2 monomer measured can include fragments thereof, degradation products thereof, and/or enzymatic cleavage products thereof.

In particular, with respect to a predetermined level as employed for monitoring disease progression and/or treatment, the amount or concentration of laminin gamma-2 monomer (e.g., laminin gamma-2 monomer, variants of laminin gamma-2 monomer, or any combinations thereof) may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for laminin gamma-2 monomer is defined in accordance with standard practice. A so-called altered level or alteration can be considered to have occurred when there is any net change as compared to the typical or normal level or range, or reference level or range, which cannot be explained by experimental error or sample variation. Thus, the level measured in a particular sample will be compared with the level or range of levels determined in similar samples from a so-called normal subject. In this context, a “normal subject” is an individual with no detectable disease or disorder, and a “normal” (sometimes termed “control”) patient or population is/are one(s) that exhibit(s) no detectable disease or disorder, respectively, for example. An “apparently normal subject” is one in which laminin gamma-2 monomer has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, cancer.

The method of assay can also involve the assay of other markers and the like as discussed herein and known in the art. For example, the method of assay can also involve the assay (detecting) of laminin gamma-2 monomer, and/or laminin gamma-2 monomer fragments, CEA, and CA19-9, for example.

The methods described herein also can be used to determine whether or not a subject has or is at risk of developing cancer (e.g., bladder cancer or colorectal cancer), such as discussed herein and known in the art. Specifically, such a method can comprise the steps of:
(a) determining the concentration or amount in a test sample from a subject of laminin gamma-2 monomer (e.g., laminin gamma-2 monomer, variants of laminin gamma-2 monomer, or any combinations thereof) using the methods described herein, or methods known in the art; and
(b) comparing the concentration or amount of laminin gamma-2 monomer (e.g., laminin gamma-2 monomer, variants of laminin gamma-2 monomer, or any combinations thereof) determined in step (a) with a predetermined level, wherein, if the concentration or amount of laminin gamma-2 monomer determined in step (a) is favorable with respect to a predetermined level, then the subject is determined not to have, or to be at risk for cancer as discussed herein and known in the art. However, if the concentration or amount of laminin gamma-2 monomer determined in step (a) is unfavorable (such as for example, increased) with respect to the predetermined level, then the subject is determined to have or to be at risk for cancer as discussed herein and known in the art.

Additionally, provided herein is method of monitoring the progression of disease in a subject. In some embodiments, the method comprises the steps of:
(a) determining the concentration or amount in a test sample from a subject of laminin gamma-2 monomer (e.g., laminin gamma-2 monomer, variants of laminin gamma-2 monomer, or any combinations thereof);
(b) determining the concentration or amount in a later test sample from the subject of laminin gamma-2 monomer; and
(c) comparing the concentration or amount of laminin gamma-2 monomer as determined in step (b) with the concentration or amount of laminin gamma-2 monomer determined in step (a), wherein if the concentration or amount determined in step (b) is unchanged or is unfavorable (such as, for example, increased) when compared to the concentration or amount of laminin gamma-2 monomer determined in step (a), then the disease in the subject is determined to have continued, progressed or worsened. By comparison, if the concentration or amount of laminin gamma-2 monomer as determined in step (b) is favorable when compared to the concentration or amount of laminin gamma-2 monomer as determined in step (a), then the disease in the subject is determined to have discontinued, regressed or improved.

As described herein, in some embodiments the various methods disclosed herein include providing any of the concentration, amount, or comparison of laminin gamma-2 monomer as determined in the various method steps. Once provided, the concentration, amount or comparison of laminin gamma-2 monomer in a sample can be used to provide a diagnosis, prognosis, or risk assessment of disease (e.g., assessment of disease progression or assessment of likelihood of developing a disease), or monitor the course of disease in a subject (e.g., in a subject undergoing treatment or a subject having been treated and monitored for recurrence of disease). Optionally, the method further comprises comparing the concentration or amount of laminin gamma-2 monomer as determined in step (b), for example, with a predetermined level. Further, optionally the method comprises treating the subject with one or more pharmaceutical compositions for a period of time if the comparison shows that the concentration or amount of laminin gamma-2 monomer as determined in step (b), for example, is unfavorably altered (such as, for example, increased) with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions, such as one or more chemotherapeutic or biologic drug. Next, the concentration or amount of laminin gamma-2 monomer is determined (e.g., using the methods described herein or as known in the art) in a first test sample from a subject. After the concentration or amount of laminin gamma-2 monomer is determined, optionally the concentration or amount of laminin gamma-2 monomer is then compared with a predetermined level. If the concentration or amount of laminin gamma-2 monomer as determined in the first test sample is lower than the predetermined level, then the subject is not treated with one or more pharmaceutical compositions. However, if the concentration or amount of laminin gamma-2 monomer as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about one (1) day to about thirty (30) days, at which time the success of the treatment can be assessed (e.g., using clinical indicators or determining the concentration or amount of laminin gamma-2 monomer after treatment has begun).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of laminin gamma-2 monomer (e.g., laminin gamma-2 monomer, variants of laminin gamma-2 monomer, or any combinations thereof) is determined in the second or subsequent test sample is determined (e.g., using the methods described herein or as known in the art). The concentration or amount of laminin gamma-2 monomer as determined in each of the second and subsequent test samples is then compared with the concentration or amount of laminin gamma-2 monomer as determined in the first test sample (e.g., the test sample that was originally optionally compared to the predetermined level). If the concentration or amount of laminin gamma-2 monomer as determined in step (c) is favorable when compared to the concentration or amount of laminin gamma-2 monomer as determined in step (a), then the disease or infection in the subject is determined to have discontinued, regressed or improved, and the subject should continue to be administered the one or pharmaceutical compositions of step (b). However, if the concentration or amount determined in step (c) is unchanged or is unfavorable (such as, for example, increased) when compared to the concentration or amount of laminin gamma-2 monomer as determined in step (a), then the disease is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received, and evaluate the efficacy of the different composition(s) to decrease or lower said subject's laminin gamma-2 monomer level and/or improve symptoms of the disease.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years, or about 10.0 years or more after the first test sample from the subject is obtained, or at least about after one of the aforementioned time periods. When used to monitor disease progression, the above assay can be used to monitor the progression of disease in subjects suffering from cancer and/or any conditions associated with cancer. Such conditions may typically chronic, when the cancer is not cured, or such conditions can be acute (also known as critical care conditions). Acute conditions are often life-threatening diseases or other critical medical conditions involving, for example, the cardiovascular, neurological, or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., repeated every about 1 minute, about 5 minutes, about 10 minutes, about 15 minutes, about 30 minutes, about 45 minutes, about 60 minutes, about 2 hours, about 3 hours, about 4 hours, 4 about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days, or at least about every one of the aforementioned timeframes, and the initial assay likewise is generally done within a shorter timeframe, e.g., about minutes, hours or days of the onset of the disease or condition.

Suitably, the assays also can be used to monitor the progression of disease in subjects suffering from chronic or non-acute conditions. Non-critical care or, non-acute conditions, refers to conditions other than acute, life-threatening disease or other critical medical conditions. Typically, non-acute conditions include those of longer-term or chronic duration. For non-acute conditions, repeat monitoring generally is done with a longer timeframe, e.g., hours, days, weeks, months or years (e.g., after about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years, or more (e.g., for the lifespan of the subject)), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as blood, urine, serum or plasma. Optionally the above assays can then be repeated using a second test sample obtained from the subject where the second test sample is obtained from the same or another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a cancer will benefit from treatment. In particular, the disclosure relates to laminin gamma-2 monomer companion diagnostic methods and products. Thus, the method of “monitoring the treatment of disease in a subject” as described herein further optimally also can encompass selecting or identifying candidates for therapy, such as therapy with chemotherapeutics, biologics, radiation, palliative care, hormone therapy, and/or surgery.

Thus, in some embodiments, the disclosure also provides a method of determining whether a subject having cancer, or at risk for having cancer (as discussed herein and known in the art) is a candidate for a particular cancer therapy. Generally, the subject is one who has experienced some symptom of the disease or who has actually been diagnosed as having, or being at risk for, cancer, and/or who demonstrates an unfavorable concentration or amount (such as, for example, an increased concentration of laminin gamma-2 monomer when compared to a predetermined level) of laminin gamma-2 monomer or a variant thereof, as described herein.

The method optionally comprises an assay as described herein, where analyte is assessed before and following treatment of a subject with one or more pharmaceutical compositions, or where analyte is assessed following such treatment and the concentration or the amount of analyte is compared against a predetermined level. An unfavorable concentration (such as, for example, an increased concentration when compared to a predetermined level) of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

While certain embodiments herein are advantageous when employed to assess cancer, or risk of cancer onset, the assays and kits also optionally can be employed to assess laminin gamma-2 monomer in other diseases, disorders and conditions as appropriate.

Generally, any method that can detect or quantify biomarkers in a sample can be used in the methods described herein. These methods include physical and molecular biology methods in addition to immunological methods. For example, suitable physical methods include mass spectrometric methods, fluorescence resonance energy transfer (FRET) assays, chromatographic assays, and dye-detection assays. Suitable molecular biology methods that can be used include, but are not limited to, Northern or Southern blot hybridization, nucleic acid dot- or slot-blot hybridization, in situ hybridization, nucleic acid chip assays, PCR, reverse transcriptase PCR (RT-PCR), or real time PCR (taq-man PCR). Other methods to detect biomarkers include, e.g., nuclear magnetic resonance (NMR), fluorometry, colorimetry, radiometry, luminometry, or other spectrometric methods, plasmon-resonance (e.g. BIACORE), and one- or two-dimensional gel electrophoresis.

Once measured, the concentration of laminin gamma-2 monomer and that of any other additional biomarker being assessed is compared to a predetermined reference value for the specific biomarker. A measured, i.e. determined, laminin gamma-2 monomer concentration that exceeds the reference laminin gamma-2 monomer value is indicative of cancer or increased risk of cancer in the subject. The reference value may be determined in one of several ways. For example, the laminin gamma-2 monomer reference value can be the laminin gamma-2 monomer concentration measured in a sample taken from a control subject, or may be the median laminin gamma-2 monomer concentration calculated from the concentrations measured in multiple control samples taken from a group of control subjects. A median laminin gamma-2 monomer concentration is preferably obtained from a group of at least 20 control subjects, at least 30 control subjects, or at least 40 control subjects. The predetermined reference value for the biomarker can be a predetermined cutoff value.

A “control subject” is a healthy subject, i.e. a subject having no clinical signs or symptoms of cancer. Preferably a control subject is clinically evaluated for otherwise undetected signs or symptoms of cancer, which evaluation may include routine physical examination and/or laboratory testing.

Alternatively, a laminin gamma-2 monomer cutoff value (or predetermined cutoff value) can be determined by a receiver operating curve (ROC) analysis from biological samples of a patient group. ROC analysis as generally known in the biological arts is a determination of the ability of a test to discriminate one condition from another, e.g. diseased cases from normal cases, or to compare the diagnostic performance of two or more laboratory or diagnostic tests. A description of ROC analysis as applied according to the present disclosure is provided in P. J. Heagerty et al., Time-dependent ROC curves for censored survival data and a diagnostic marker, Biometrics 56:337-44 (2000), the disclosure of which is hereby incorporated by reference in its entirety. Alternatively, a laminin gamma-2 monomer cutoff value can be determined by a quartile analysis of biological samples of a patient group. For example, a laminin gamma-2 monomer cutoff value can be determined by selecting a value that corresponds to any value in the 25th-75th percentile range, preferably a value that corresponds to the 25th percentile, the 50th percentile or the 75th percentile, and more preferably the 75th percentile. Still further alternatively, a laminin gamma-2 monomer cutoff value can be determined by a mean plus two standard deviation analysis of biological samples of a patient group. An exemplary laminin gamma-2 monomer reference value obtained from the median of a relevant patient group is about 950 pg/ml in serum. An exemplary laminin gamma-2 monomer reference value obtained from quartile analysis at the 75th percentile is about 1200 pg/ml in serum. Such statistical analyses can be performed using any method known in the art and can be implemented through any number of commercially available software packages (e.g., from Analyse-it Software Ltd., Leeds, U K; StataCorp LP, College Station, Tex.; SAS Institute Inc., Cary, N.C.). An exemplary laminin gamma-2 monomer reference value obtained from a mean plus two standard deviation analysis is about 1050 pg/ml in serum.

In some embodiments, the methods comprise a laminin gamma-2 monomer cutoff value of about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, or about 1,000 pg/mL.

The method may further include assessing at least one additional biomarker of cancer, for example by measuring the concentration at least one additional biomarker in the biological sample, and comparing the measured concentration to a reference value for each additional biomarker being assessed. One, two, three, four or more additional biomarkers may be assessed. Additional such biomarkers of cancer include, but are not limited to, CEA, CA19-9, and fragments of laminin gamma-2 monomer (e.g., the EGF-like fragment generated by MT1-MMP). A reference value (or predetermined level) may be similarly determined for any other biomarker of cancer, as described herein with respect to determining a reference value for laminin gamma-2 monomer. Typically, a measured i.e., determined concentration of any additional biomarker in a biological sample that exceeds the reference value for that biomarker is also indicative of cancer or increased risk of cancer onset in the subject. Instances of biomarkers for which the opposite is true are nevertheless possible, i.e. biomarkers for which the relationship between concentration in a biological sample and instance of cancer or increased risk of cancer onset is inverse, such that a determined biomarker concentration that is below the reference value for the biomarker is indicative of cancer or increased risk of cancer onset in the subject.

For example, elevated levels CEA in the blood have been used as diagnostic biomarkers of cancer, such as colorectal cancer. CEA is often evaluated in patients suspected of having some forms of cancer even though positive results can be due to other causes, and negative results do not rule out disease. Nevertheless, in combination with signs and symptoms, CEA can play a role in both diagnosis and disease prognosis, and is part of the usual disease diagnostic criteria of some cancer types. Increased levels of CEA can occur in cancer. Typically, higher levels of CEA in a sample correlate to a greater probability of the presence of, or onset of, cancer. Thus, embodiments of the methods described herein comprise determining the concentration of CEA (and/or CA 9-9, fragments of laminin gamma-2 monomer, etc.) and laminin gamma-2 monomer in a sample.

Thus, in some embodiments the methods encompass detection of laminin gamma-2 monomer and at least one marker selected from CEA, CA19-9, and fragments of laminin gamma-2 monomer. In some embodiments, the methods encompass detection of laminin gamma-2 monomer and CEA, and optionally, at least one marker selected from CA19-9 and fragments of laminin gamma-2 monomer. In some embodiments, the methods encompass detection of laminin gamma-2 monomer and CA19-9 and, optionally, at least one marker selected from CEA and fragments of laminin gamma-2 monomer. In some embodiments, the methods encompass detection of laminin gamma-2 monomer, and fragments of laminin gamma-2 monomer and, optionally, at least one marker selected from CEA and CA19-9. In some embodiments, the methods encompass detection of laminin gamma-2 monomer, CEA, CA 19-9, and fragments of laminin gamma-2 monomer.

### C. KITS

Provided herein is a kit, which may be used for treating a subject suffering from cancer, or at increased risk of cancer, or diagnosing a subject as having cancer as described previously herein.

Kits to be used for treating a patient will contain an antibody specific for laminin gamma-2 monomer. The kits preferably include instructions for treating a subject using the antibodies described herein. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Also provided is a kit for assaying a test sample for laminin gamma-2 monomer (e.g., laminin gamma-2 monomer, variants of laminin gamma-2 monomer, or any combinations thereof). The kit comprises at least one component for assaying the test sample for laminin gamma-2 monomer and instructions for assaying the test sample for (e.g., laminin gamma-2 monomer, variants of laminin gamma-2 monomer, or any combinations thereof). The at least one component includes at least one composition comprising an isolated antibody that specifically binds to (e.g., laminin gamma-2 monomer, variants of laminin gamma-2 monomer, or any combinations thereof). The antibody has a variably heavy domain region and a variable light domain region. The antibody is optionally detectably labeled.

For example, the kit can comprise instructions for assaying the test sample for (e.g., laminin gamma-2 monomer, variants of laminin gamma-2 monomer, or any combinations thereof) by immunoassay, e.g., chemiluminescent microparticle immunoassay. The instructions can be in paper form or computer-readable form, such as a disk, CD, DVD, or the like. The antibody can be a laminin gamma-2 monomer capture antibody and/or a laminin gamma-2 monomer detection antibody. Alternatively or additionally, the kit can comprise a calibrator or control, e.g., purified, and optionally lyophilized, (e.g., laminin gamma-2 monomer, variants of laminin gamma-2 monomer, or any combinations thereof), and/or at least one container (e.g., tube, microtiter plates or strips, which can be already coated with an anti-laminin gamma-2 monomer monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying laminin gamma-2 monomer.

Any antibodies, which are provided in the kit, such as recombinant antibodies specific for laminin gamma-2 monomer, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of cancer. Examples of analytes include, but are not limited to laminin gamma-2 monomer, CEA, CA19-9, and fragments of laminin gamma-2 monomer as well other analytes and biomarkers discussed herein, or otherwise known in the art. In some embodiments one or more components for assaying a test sample for laminin gamma-2 monomer enable the determination of the presence, amount or concentration of laminin gamma-2 monomer. A sample, such as a serum sample, can also be assayed for laminin gamma-2 monomer using TOF-MS and an internal standard.

The kit (or components thereof), as well as the method of determining the concentration of laminin gamma-2 monomer in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing laminin gamma-2 monomer is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, laminin gamma-2 monomer, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of laminin gamma-2 monomer in the sample by means of an embedded algorithm and factory-determined calibration curve.

It will be understood that the methods and kits as described herein necessarily encompass other reagents and methods for carrying out the immunoassay. For instance, the disclosure encompasses various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an I-STAT® cartridge format, using a nucleic acid sequence linked to the signal antibody as a signal amplifier.

If desired, multiple concentrations of each antibody can be included in the kit to facilitate the generation of a standard curve to which the signal detected in the test sample can be compared. Alternatively, a standard curve can be generated by preparing dilutions of a single antibody solution provided in the kit.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

### EXAMPLES

Materials and Methods

Rabbit polyclonal antibody used in the examples was established by immunizing a rabbit with purified domain III of human laminin gamma-2 chain that was recombinantly expressed in E. coli. (Domain III (383-608 aa) was expressed as a GST fusion protein using Gateway technology (Invitrogen, Grand Island, N.Y.)). Mouse monoclonal antibody 2H2 was provided from Dr. Koshikawa and Dr. Seiki from the University of Tokyo (Monoclonal antibody 2H2 is described in Koshikawa N, et al. Cancer Res. 2008; 68: (2). Jan. 15, 2008). Monoclonal antibodies D4B5 and 1H3 (D4B5 is commercially available from Millipore, Billerica, Mass. 1H3 is described in Koshikawa N, et al. Cancer Res. 2008: 68: (2). Jan. 15, 2008). Specimens from bladder cancer patients were purchased from ProMedDx (Norton, Mass.). Specimens from colorectal cancer patients were purchased from ProMedDx (Norton, Mass.) and KAC Co., Ltd. (Kyoto, Japan). Specimens from pancreatic cancer patients were purchased from ProMedDx (Norton, Mass.) Bioreclamation LLC (Long Island, N.Y.) and KAC Co., Ltd. (Kyoto, Japan). Specimens from ovarian cancer patients were purchased from Bioreclamation LLC (Long Island, N.Y.) and KAC Co., Ltd. (Kyoto, Japan). Specimens from stomach cancer patients were purchased from ProMedDx (Norton, Mass.) and KAC Co., Ltd. (Kyoto, Japan). Specimens from esophagus cancer patients were purchased from Bioreclamation LLC (Long Island, N.Y.). A total of 109 normal specimens were purchased from several vendors including KAC Co., Ltd. (Kyoto, Japan), Bioreclarmation LLC (Long Island, N.Y.), SeraCare life Sciences Inc. (Milford, Mass.) and Complex Antibodies, Inc (Fort Lauderdale, Fla.).

### Example 1

### Validation of Antibodies & Prototype ELISA

A series of experiments were performed in order to characterize the binding properties of various antibodies for use in establishing an exemplary ELISA for the laminin gamma-2 monomer.

Binding Specificity of Monoclonal Antibody 2H2 to Gamma-2 Monomer

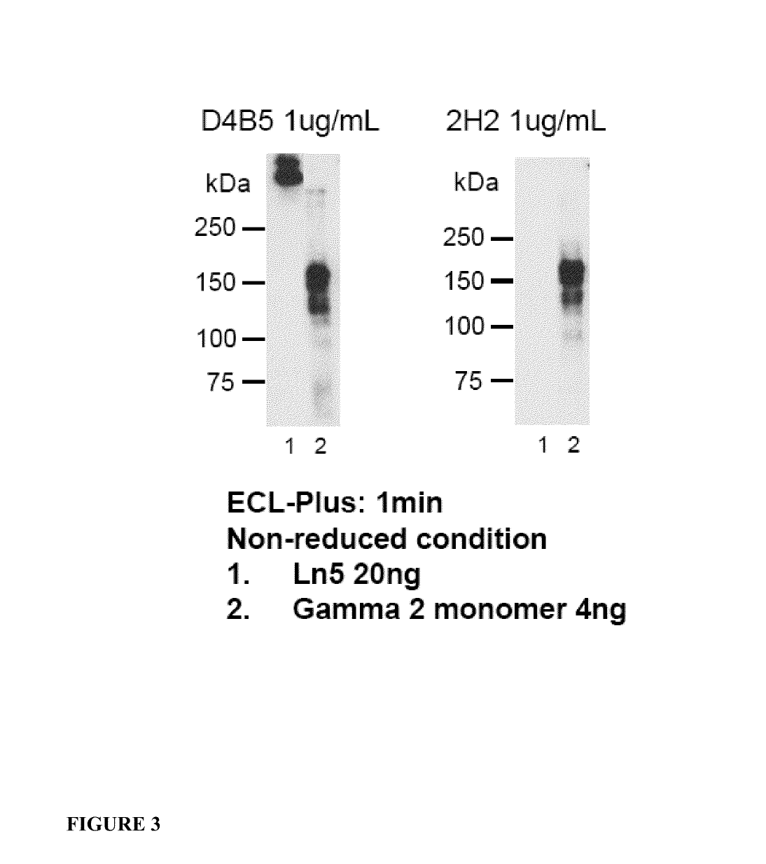

Western blot assays were performed in order to evaluate the specificity of monoclonal antibody 2H2 to gamma-2 monomer and the degree of its cross-reactivity with laminin 5. As illustrated in FIG. 3, antibody monoclonal D4B5 (Purchased from Millipore, Billerica, Mass.) exhibits binding activity to both the laminin 5 complex (at 20 ng; Lane 1 of FIG. 3) and gamma-2 monomer (at 4 ng: Lane 2 of FIG. 3). In contrast monoclonal antibody 2H2 shows binding specificity to gamma-2 monomer without any binding activity to the laminin 5 complex. Each monoclonal antibody was used at a concentration of 1 μg/mL.

Spike Recovery and Dilution Linearity

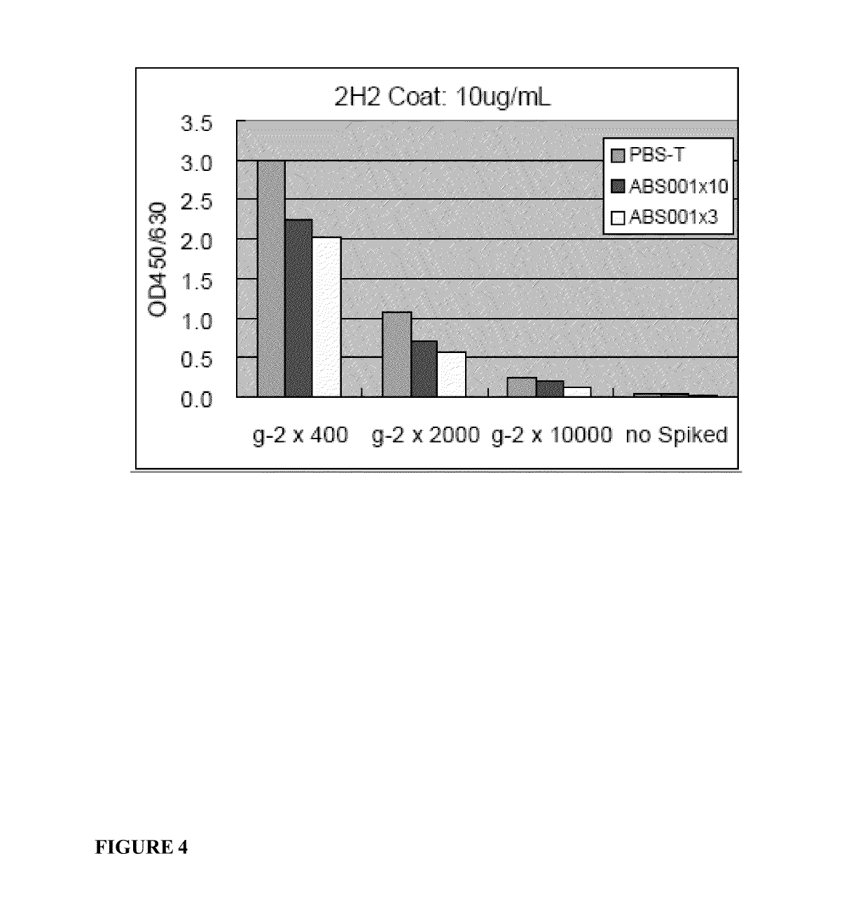

To assess antibody performance in dilution linearity, monoclonal antibodies D4B5 (5 μg/mL), 1H3 (10 μg/mL), and 2H2 (10 μg/mL) (Sources D4B5 was purchased from D4B5 is commercially available from Millipore, Billerica, Mass. 2H2 and 1H3 were provided from Dr. Koshikawa and Dr. Seiki (University of Tokyo) and described in Koshikawa N, et al. Cancer Res. 2008; 68: (2). Jan. 15, 2008) were coated in the wells of a 96-well microtiter plate (Costar) using standard procedures. Briefly, the wells were coated with antibody at various concentrations overnight in phosphate buffered saline (PBS) at 4° C. The wells were blocked with 3% bovine serum albumin (BSA) for 1 hr at 37° C. Recombinant laminin gamma-2 monomer at various dilutions (400×, 2000×, 10000×, and no addition/spike) was added to diluent (PBS) and to two different dilutions (3× and 10×) of a normal serum specimen sample (Complex Antibodies, Inc.). Each of the antibodies showed good response linearity to the dilutions in laminin gamma-2 monomer as well as dilutions in matrix (i.e., normal serum sample). An illustrative example of the data from this experiment is shown for the 2H2 monoclonal antibody in FIG. 4.

The monoclonal antibody 2H2 was coated at 10 μg/mL in a 96-well microtiter plate and further evaluated using a spike recovery assay. Recombinant laminin gamma-2 monomer was spiked in diluent (PBS) and in a normal serum specimen sample (ProMedDx). The target concentration range of laminin gamma-2 monomer for this experiment spanned from 0-20 ng/mL (0.00, 0.31, 0.63, 1.25, 2.50, 5.00, 10.00, and 20.00 ng/mL). Once the concentration in the normal specimen sample was adjusted for endogenous signal (i.e., no spike) the mean spiked recovery of laminin gamma-2 monomer was calculated as 86.3%.

Establishing Sensitivity of ELISA Using 2H2 as Capture Antibody

An ELISA assay was performed using the 2H2 monoclonal antibody as the capture antibody, the rabbit polyclonal antibody to domain III of human laminin gamma-2 as the detection antibody, and a secondary anti-rabbit antibody conjugated to horseradish peroxidase in order to assess the analytical sensitivity and reliable concentration range of the laminin gamma-2 monomer ELISA. Briefly, wells in a 96-well plate were coated with 50 μL of 2H1-2 antibody (10 μg/mL) overnight at 4° C. The wells were washed three times with 200 μL/well of PBS. The wells were blocked with 200 μL of a solution containing of BSA (3%) in PBS for 1 hr at 37° C. Following three washes with 200 μL of 0.1% Tween-20 in PBS, the plates were stored at −20° C. until use.

For the ELISA, the following solutions were prepared:

Sample solutions were prepared as 10× specimen dilution in PBS containing 1% BSA, 10 mM EDTA, 2% Tween 20, 0.2 mg/mL HBR.

Wash solution was PBS containing 0.1% Tween-20.

Detection antibody solution (or “first” antibody solution) contained polyclonal rabbit antibody in PBS containing 1% BSA, 10 mM EDTA, 2% Tween-20, 0.2 mg/mL HBR. This solution was added to the wells (50 μL) at room temperature and incubated for 1 hr.

Secondary antibody solution (or “second” antibody solution) contained either a F(ab′)2 fragment of donkey anti-rabbit IgG antibody or goat anti-rabbit IgG conjugated to horseradish peroxidase (HRP) in 5000× excess in PBS containing 1% BSA, 10 mM EDTA, 2% Tween-20, 0.2 mg/mL HBR.

ELISA Protocol: To the wells, 50 μL of the sample solution were added and allowed to incubate for 2 hr at 37° C. The wells were washed with wash solution 3× with 200 μL/wash. Following the wash, detection antibody solution was added to the wells (50 μL/well) at room temperature and incubated for 1 hr. The wells were again washed 3× with 200 μL wash solution per wash. The secondary antibody solution (50 μL/well) was added to the wells at room temperature and incubated for 1 hr. Following four rounds of washes (200 μL/wash), 100 μL of tetramethyl benzidine (TMB) was added to each well. The reaction was stopped by adding 100 μL/well of 0.6% sulfuric acid solution, incubated for 20 min. OD readings were made at 450 and 630 nm (Bio-Rad).

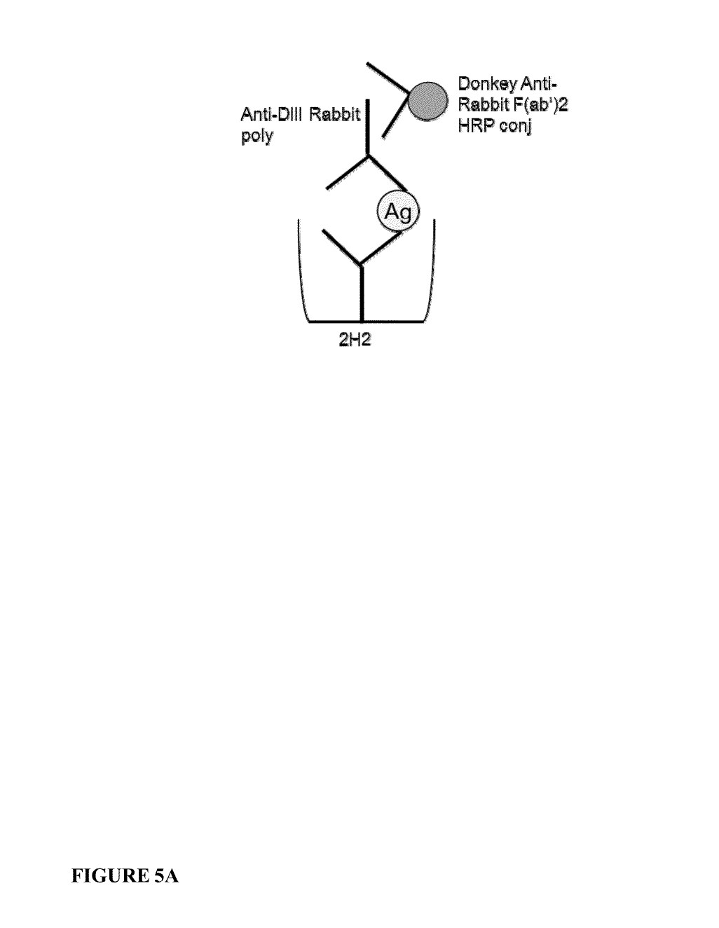

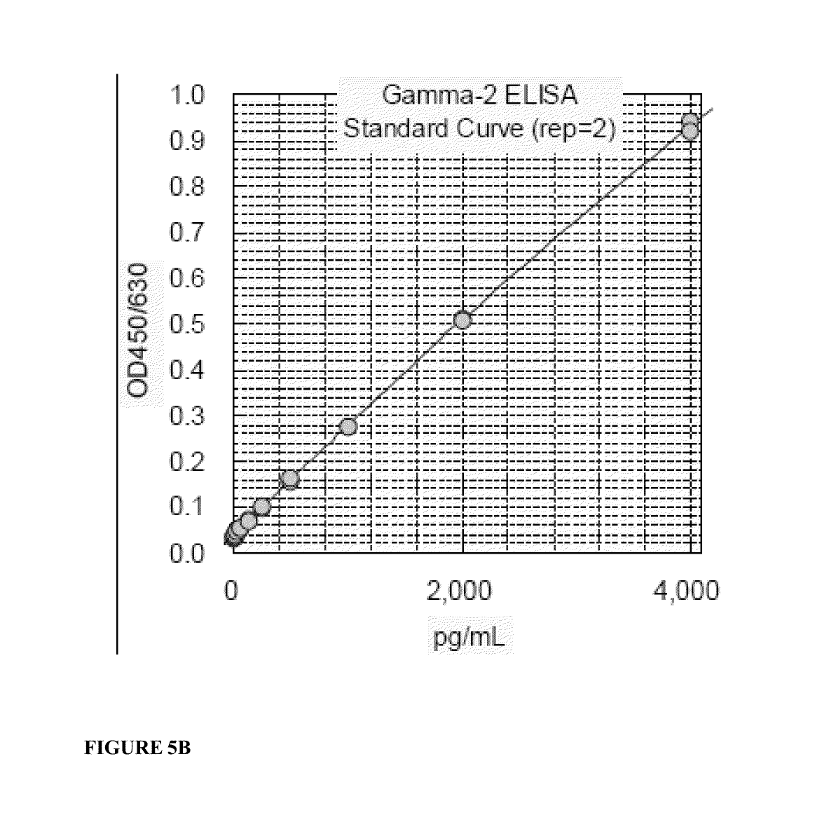

As shown in FIG. 5B, the established standard curves demonstrate the analytical sensitivity of the assay to be about 3.7 pg/mL, making the reliable detection limit for this ELISA to be about 4 pg/mL, and the linear detection range to be at least 0-4,000 μg/mL.

Dilution Linearity Analysis Using Laminin Gamma-2 Monomer Spiked Cancer Specimens and Normal Specimens

Further evaluation of the dilution linearity was performed using two cancer specimens (one bladder cancer specimen, one pancreatic cancer specimen) and two normal specimens. The normal specimens were spiked with recombinant laminin gamma-2 monomer. For dilution factors ranging from 1:8 to 1:1 (i.e., 8× to 1× dilution) the results demonstrated a recovery of 95-116% (versus neat) in the cancer specimens and 93-113% in normal specimens.

### Example 2

### Measurement of Laminin Gamma-2 Monomer

Ten serums specimens from patients with bladder cancer and twenty five serum specimens from patients with colorectal cancer were prepared along with normal specimens (109) for determination of various biomarker concentrations. The measurements of the samples were performed using the ARCHITECT system for CEA and CA19-9, while laminin gamma-2 monomer concentration was determined using the ELISA assay detailed above. All commercial kits were used according to the manufacturer's instructions.

Results

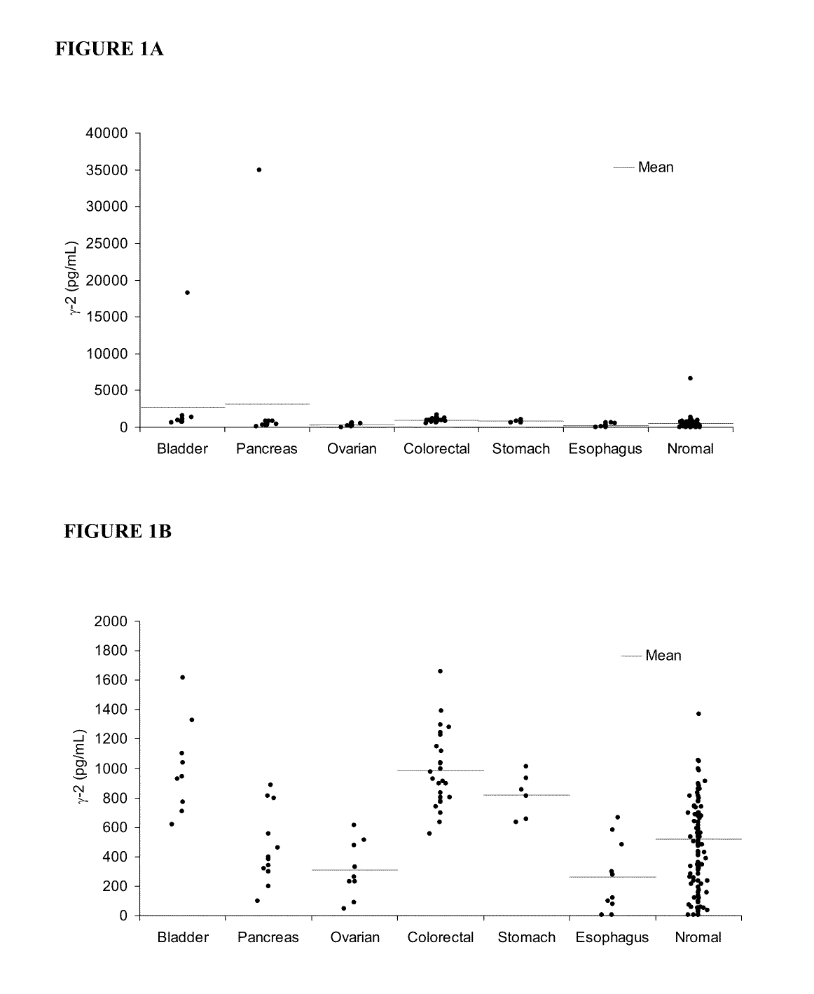

FIG. 1 depicts dot plots of levels of laminin gamma-2 monomer in serum samples from patients with various cancers, including bladder cancer (n=10) and colorectal cancer (n=25), as well as serum samples from healthy control patients. [The median levels of laminin gamma-2 monomer in from patients with bladder cancer were 992 pg/mL, for patients with colorectal cancer 929 pg/mL, and for the healthy controls 485 pg/mL. Using the student's t-test: bladder cancer versus healthy control was p=0.000041, colorectal cancer versus healthy control was p=0.00031] The concentration of laminin gamma-2 monomer was significantly higher in serum samples from specimens from the bladder and colorectal cancer specimens when compared to concentration of laminin gamma-2 monomer in the normal specimens or the other cancer specimens (e.g., pancreas, ovarian, stomach, and esophagus).

LN Gamma-2 Monomer—Sensitive and Specific Serum Marker for Bladder and Colorectal Cancer

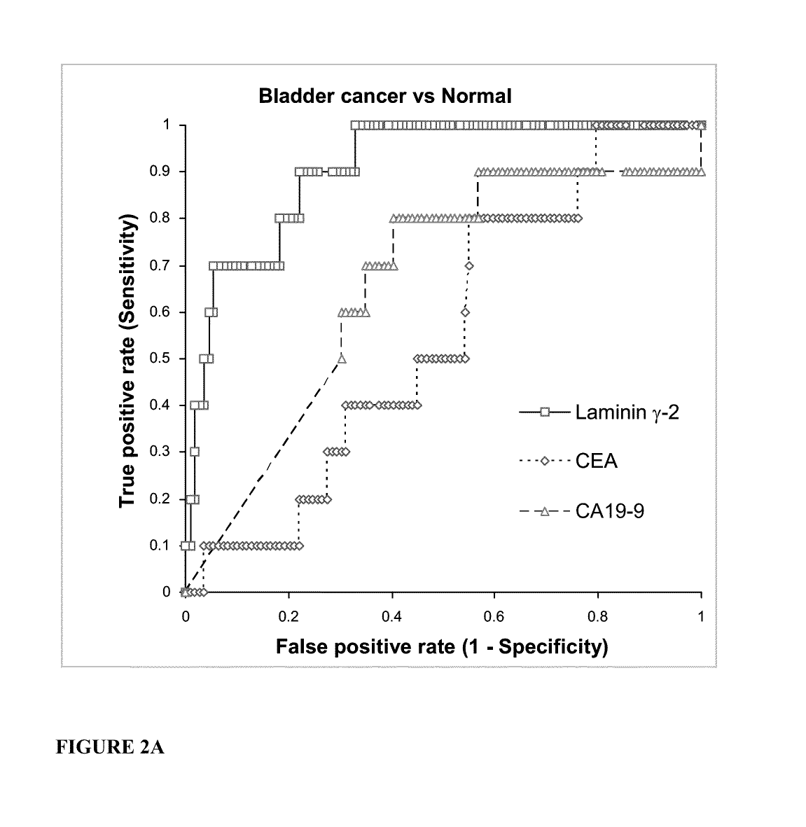

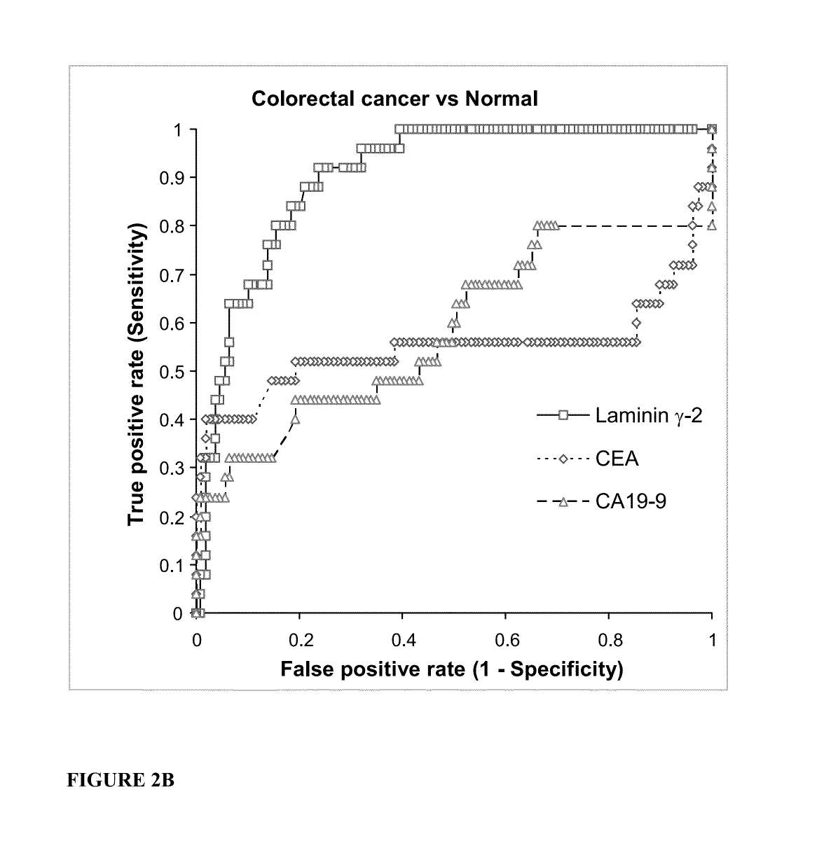

A Receiver Operating Characteristic (ROC) plot was generated from the observed true positive rate of the three measured biomarker (laminin gamma-2 monomer, CEA, CA19-9) levels in the bladder cancer and colorectal cancer specimens against the observed false positive rate of the three biomarkers levels in normal specimens (FIG. 2). A diagonal line from the lower left axis across the plot to the upper right corner (e.g., coordinate of (1,1)) would be indicative of a plot having the worst possible prediction method in which marker levels would not discriminate at all between cancer specimens and normal specimens. A best possible prediction method is expected to yield a point in the upper left corner or coordinate (0,1) of the ROC space, representing 100% sensitivity (no false negatives) and 100% specificity (no false positives). Thus, an area under the curve (AUC) derived from a plot of actual data which approaches the value of 1.0 represents a best possible prediction method. As shown in FIG. 2, the ROC plot curves demonstrate the sensitivity and specificity of the laminin gamma-2 monomer marker for both bladder cancer and colorectal cancer when compared to other biomarkers associated with bladder and colorectal cancer diagnosis (CEA and CA19-9). From this data laminin gamma-2 monomer demonstrates superior sensitivity and specificity as a marker for bladder cancer and colorectal cancer, relative to the existing biomarkers for those diseases.

TABLE 1
ROC Summary (Bladder cancer v. Normal).

| Marker | Area | 95% CI | SE |
|---|---|---|---|
| Laminin γ2 monomer | 0.91 | 0.83 to 0.99 | 0.039 |
| CEA | 0.55 | 0.29 to 0.61 | 0.083 |
| CA19-9 | 0.66 | 0.48 to 0.84 | 0.091 |

TABLE 2
ROC Summary (Colorectal cancer v. Normal).

| Marker | Area | 95% CI | SE |
|---|---|---|---|
| Laminin γ2 monomer | 0.90 | 0.85 to 0.96 | 0.028 |
| CEA | 0.55 | 0.37 to 0.73 | 0.092 |
| CA19-9 | 0.58 | 0.43 to 0.74 | 0.077 |

This data establishes for the first time, a) the ability to detect laminin gamma-2 monomer in serum, and b) the unexpected association of increased concentration of laminin gamma-2 monomer in serum (compared to concentrations in samples from normal (healthy) controls as well as other cancer types) with the occurrence of bladder cancer and colorectal cancer in patient samples. Moreover the data also established that laminin gamma-2 monomer shows superior diagnostic accuracy for both bladder cancer and colorectal cancer when compared with other existing and clinically relevant biomarkers. The disclosure also establishes for the first time a diagnostic test that can be used to detect laminin gamma-2 monomer in serum with an increased sensitivity and specificity (i.e., it does not detect laminin 5) and does not require proteolytic processing of laminin gamma-2 monomer for detection (i.e., the assay is not specific to the laminin gamma-2 N-terminal fragment generated by MT1-MMP).

Thus, laminin gamma-2 monomer can be used to diagnose cancer such as, bladder cancer and colorectal cancer, in patient samples, or to provide a prognosis for risk or progression of cancer, such as bladder cancer and colorectal cancer, in a subject or patient. Similarly, elevated levels of laminin gamma-2 monomer can be used to identify a patient as a candidate for therapy comprising one or more cancer therapies.

### Example 3

### Establishing Prototype Reagents for the Auto-Immunoassay Instrument ARCHITECT™

A series of experiments was performed in order to transfer the ELISA reagents into auto-immunoassay reagents for the detection of the laminin gamma-2 monomer.

The monoclonal antibody 2H2 was coated on the Paramagnetic Microspheres (Varian Medical Systems, Palo Alto, Calif.). The carboxyl group modified microparticles were then washed with MES buffer (pH5.5) and then the MES buffer containing N-(3-Dimethylaminopropyl)-N′-ethyl-carbodiimidehydrochloride (SIGMA-ALDRICH, St Louis, Mo.) and N-Hydroxy-succinimide (SIGMA-ALDRICH, St Louis, Mo.) were added to the microparticles. After a 30 min incubation at room temperature, the reagents were washed out and the antibody diluted with MES buffer was added to the microparticle. The final concentration of the antibody in this reaction was 0.3 mg/mL. After 2 hrs incubation in room temperature, microparticles were washed with TBS with 1% Tween-20 and stored at 2-8 Celsius.

The rabbit polyclonal antibody to domain III of human laminin gamma-2 (used as the detection antibody in ELISA) was conjugated with Acridinium in PBS buffer containing 0.5% CHAPS. The excess acridinium was removed by Zeba Micro Desalt Spin Columns (Thermo Fisher Scientific, Waltham, Mass.). The assay sample diluent was prepared based on phosphate buffer with 1% BSA and 0.1% Tween-20.

The ARCHITECT™ assay was performed on an i2000 ARCHITECT™ analyzer. Recombinant laminin gamma-2 monomer was spiked in sample diluent (PBS with 1% BSA and 0.1% Tween-20). The calibrator range of laminin gamma-2 monomer for this experiment spanned from 0-20 ng/mL (0.00, 0.01, 0.02, 0.10, 1.00, 10.00 and 20.00 ng/mL).

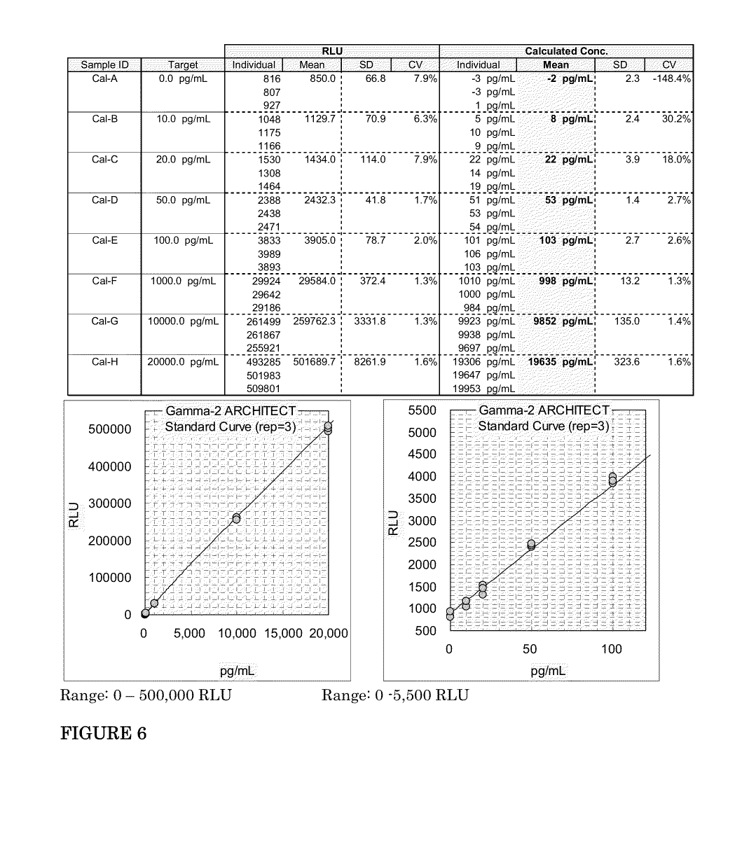

As shown in FIG. 6, the preliminary standard curves demonstrate the analytical sensitivity of the assay to be about or approximately 10 pg/mL. Thus, the assay is quite sensitive.

Dilution Linearity Analysis Using Laminin Gamma-2 Monomer Spiked Cancer Specimens and Normal Specimens

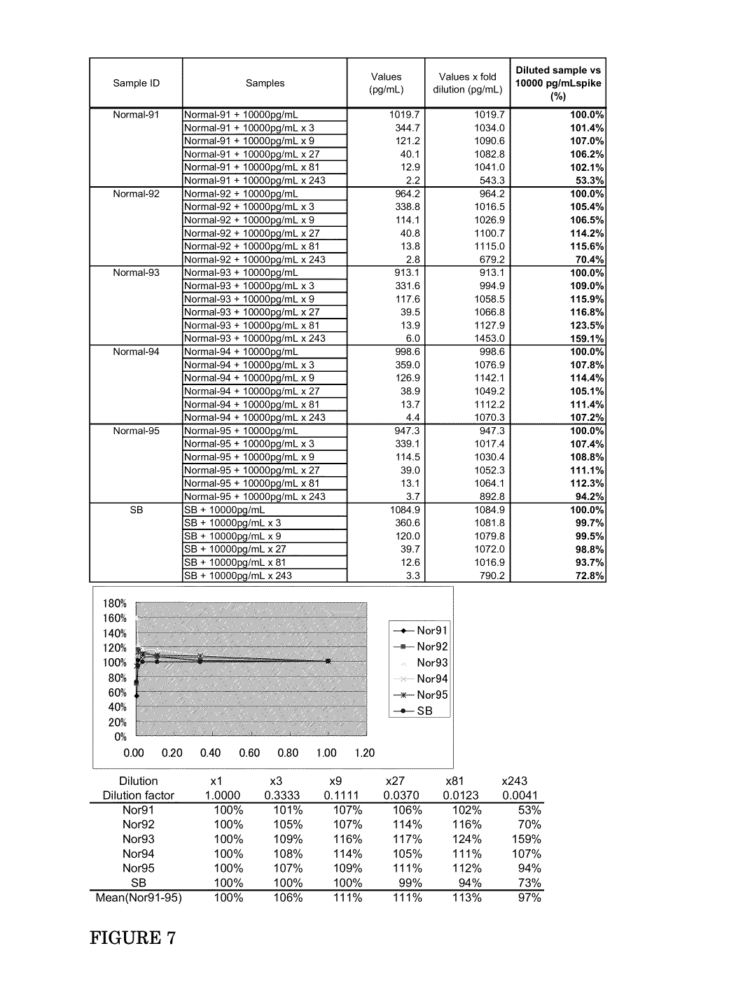

Further evaluation of the dilution linearity was performed using five normal specimens. The normal specimens were spiked with recombinant laminin gamma-2 monomer. The spiked specimens were diluted with sample diluent to produce a three-fold dilution series. As shown on FIG. 7, the dilution factors ranged from 1:81 to 1:3 (i.e., 81×to 3× dilution). The results demonstrated an average recovery of 100-113% (versus neat) in 5 normal specimens. The preliminary assay on ARCHITECT™ showed good performance with respect to the dilution linearity testing.

Evaluation of the Commercially Available Normal Specimens

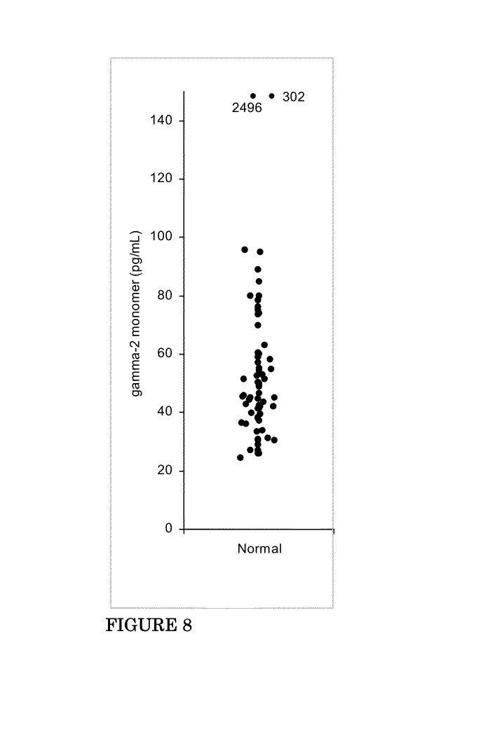

A total of 66 normal specimens were purchased from several vendors including ProMedDx (Norton, Mass.), KAC Co., Ltd. (Kyoto, Japan), SeraCare life Sciences Inc. (Milford, Mass.) and Complex Antibodies, Inc (Fort Lauderdale, Fla.). The laminin gamma-2 monomer level in normal specimens was measured by ARCHITECT™. The results were shown in FIG. 8. A median value of normal specimens was 46.0 pg/mL, and the 95 percentile was 95.0 pg/mL. A mean value (64 normal specimens: excluded 2 specimens, 2496 pg/mL and 302 pg/mL) was 50.2 pg/mL. From the standard deviation analysis (excluded 2 specimens, 2496 pg/mL and 302 pg/mL), the standard deviation value was 17.8 pg/ml and a mean plus two standard deviation (an appropriate cutoff value) was 85.8 pg/mL.

### REFERENCES

Koshikawa, N. et al., Overexpression of Laminin γ2 Chain Monomer in Invading Gastric Carcinoma Cells; Cancer Res (1999) 59:5596-5601.
Koshikawa, N. et al., Development of a New Tracking Tool for the Human Monomeric Laminin-γ2 Chain In vitro and In vivo; Cancer Res (2008) 68:530-536.
Koshikawa, et al., Role of Cell Surface Metalloprotease MT1-MMP in Epithelial Cell Migration over Laminin-5; J. Cell Biol., (2000) 148:615-624.
WO03016907 (EISAI CO. LTD) Reagent For Assaying Laminin 5 Antigen In Biological Sample And Assay Method.
JP2011-209281 (University of Tokyo; University Kochi) Examination Method and Examination Kit for Urological Cancer

1: A method for providing a diagnosis, prognosis or risk classification to a subject who has cancer or who is at risk of having cancer, the method comprising the steps of:
a. obtaining a biological sample comprising blood from the subject;
b. determining the concentration of laminin gamma-2 monomer in the biological sample from the subject;
c. comparing the laminin gamma-2 monomer concentration from the sample to a reference laminin gamma-2 monomer concentration value, wherein a laminin gamma-2 monomer concentration in the sample greater than the reference laminin gamma-2 monomer concentration value identifies the subject as having cancer or as having an increased risk of developing cancer.

2: A method for providing a cancer diagnosis, cancer prognosis or cancer risk classification to a subject, the method comprising the steps of:
a. obtaining a biological sample comprising blood from the subject;
b. determining the concentration of laminin gamma-2 monomer in the biological sample from the subject;
c. comparing the laminin gamma-2 monomer concentration from the sample to a reference laminin gamma-2 monomer concentration value, wherein a laminin gamma-2 monomer concentration in the sample greater than the reference laminin gamma-2 monomer concentration value identifies the subject as having cancer or as having an increased risk of developing cancer.

3: A method for providing a diagnosis, prognosis or risk classification to a subject who has cancer or who is at risk of having cancer, the method comprising the steps of:
a. obtaining a biological sample comprising blood from the subject;
b. determining the concentration of laminin gamma-2 monomer in the biological sample from the subject; and
c. providing the concentration determined from (b), to identify the subject as having cancer or having an increased risk of developing cancer when compared to a reference laminin gamma-2 monomer concentration value.

4: A method for providing a diagnosis, prognosis or risk classification to a subject who has cancer or who is at risk of having cancer, the method comprising the steps of:
a. obtaining a biological sample comprising blood from the subject;
b. determining the concentration of laminin gamma-2 monomer in the biological sample from the subject;
c. comparing the laminin gamma-2 monomer concentration from the sample to a reference laminin gamma-2 monomer concentration value;
d. providing the comparison from (c), wherein when the comparison comprises a laminin gamma-2 monomer concentration in the sample greater than the reference laminin gamma-2 monomer concentration value the comparison identifies the subject as having cancer or having an increased risk of developing cancer.

5: A method for providing a cancer diagnosis, cancer prognosis or cancer risk classification to a subject, the method comprising the steps of:
a. obtaining a biological sample comprising blood from the subject;
b. determining the concentration of laminin gamma-2 monomer in the biological sample from the subject;
c. comparing the laminin gamma-2 monomer concentration from the sample to a reference laminin gamma-2 monomer concentration value;
d. providing the comparison from (c), wherein when the comparison comprises a laminin gamma-2 monomer concentration in the sample greater than the reference laminin gamma-2 monomer concentration value, the comparison identifies the subject as having cancer or having an increased risk of developing cancer.

6: A method for detecting cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding;
wherein cancer is detected in the subject when the concentration of laminin gamma-2 monomer in the sample from the subject is higher relative to a reference laminin gamma-2 monomer concentration.

7: A method for detecting cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding; and
b. comparing the concentration of laminin gamma-2 monomer in the from the subject to a reference laminin gamma-2 monomer concentration;
wherein cancer is detected in the subject when the concentration of laminin gamma-2 monomer in the sample from the subject is higher relative to the reference laminin gamma-2 monomer concentration.

8: A method for diagnosing cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding;
wherein cancer is diagnosed in the subject when the concentration of laminin gamma-2 monomer in the sample from the subject is higher relative to a reference laminin gamma-2 monomer concentration.

9: A method for diagnosing cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding; and
b. comparing the concentration of laminin gamma-2 monomer in the from the subject to a reference laminin gamma-2 monomer concentration;
wherein when the concentration of laminin gamma-2 monomer in the sample from the subject is higher relative to the reference laminin gamma-2 monomer concentration, the concentration of laminin gamma-2 monomer in the sample indicates a diagnosis of cancer.

10: A method for prognosing cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding;
wherein the subject has a prognosis of cancer when the concentration of laminin gamma-2 monomer in the sample from the subject is higher relative to a reference laminin gamma-2 monomer concentration.

11: A method for prognosing cancer in a subject comprising:
a. determining the concentration of laminin gamma-2 monomer in a biological sample comprising blood from the subject, wherein the concentration of laminin gamma-2 monomer is determined by contacting an antibody that specifically binds to laminin gamma-2 monomer with the sample and detecting antibody binding; and
b. comparing the concentration of laminin gamma-2 monomer in the from the subject to a reference laminin gamma-2 monomer concentration;
wherein when the concentration of laminin gamma-2 monomer in the sample from the subject is higher relative to the reference laminin gamma-2 monomer concentration, the concentration of laminin gamma-2 monomer in the sample indicates a prognosis of cancer.

12: The method of any of claims 1-11, further comprising determining the concentration of at least one additional biomarker of cancer in the sample; and comparing the concentration of the at least one additional biomarker to a reference concentration value for the at least one biomarker, wherein said at least one additional marker is selected from the group consisting of a laminin gamma-2 fragment, carcinoembryonic antigen (CEA), and carbohydrate antigen 19-9 (CA19-9).

13-14. (canceled)

15: The method of any of claims 1-11, wherein the reference laminin gamma-2 monomer concentration value is a laminin gamma-2 monomer concentration value of a control sample, or a laminin gamma-2 monomer cutoff value.

16: The method of claim 15, wherein the reference laminin gamma-2 monomer concentration value is the laminin gamma-2 monomer concentration of a control sample selected from a biological sample of a control subject and a laminin gamma-2 monomer concentration standard.

17: The method of claim 15, wherein the reference laminin gamma-2 monomer concentration value is selected from the laminin gamma-2 monomer concentration value of a control sample, and the median or the mean laminin gamma-2 monomer concentration of a plurality of control samples from a group of control subjects.

18: The method of claim 15, wherein the laminin gamma-2 monomer concentration in the sample is at least double the reference laminin gamma-2 monomer concentration value.

19: The method of claim 15, wherein the reference laminin gamma-2 monomer concentration value is the laminin gamma-2 monomer cutoff value determined by a receiver operating curve (ROC) analysis from biological samples of a patient group.

20: The method of claim 15, wherein the reference laminin gamma-2 monomer concentration value is the laminin gamma-2 monomer cutoff value determined by a mean plus 2 standard deviation analysis of biological samples of a patient group.

21: The method of claim 15, wherein the reference laminin gamma-2 monomer concentration value is the laminin gamma-2 monomer cutoff value, and is about 1000 pg/mL.

22. (canceled)

23: The method of any of claims 1-5 or 10-11, wherein the method comprises providing a prognosis selected from determining the severity or stage of cancer in the subject and a likelihood that the subject will develop cancer.

24: The method of any of claims 1-11, wherein the subject is a human.

25: The method of claim 15, wherein the reference laminin gamma-2 monomer concentration value is from a biological sample comprising blood from a human control subject.

26: The method of any claims 1-11, wherein the biological sample comprising blood from the subject is selected from whole blood, plasma, and serum.

27. (canceled)

28: The method of any of claims 1-11, wherein determining the concentration of laminin gamma-2 monomer comprises an immunological method with molecules that can bind specifically to the laminin gamma-2 monomer.

29: The method of claim 28, wherein the molecules that can bind to the laminin gamma-2 monomer comprises at least one antibody capable of specifically binding laminin gamma-2 monomer.

30: The method of claim 29, wherein the molecules that can bind to the laminin gamma-2 monomer do not bind to an N-terminal fragment of laminin gamma-2.

31: A kit for performing the method of any of claims 1-11, the kit comprising:
a. at least one reagent capable of specifically binding laminin gamma-2 monomer that allows for quantifying the laminin gamma-2 monomer concentration in the biological sample; and
b. a reference standard indicating a reference laminin gamma-2 monomer concentration.

32: The kit of claim 31, further comprising at least one additional reagent capable of binding at least one additional biomarker of cancer in the biological sample that allows for quantifying the concentration of the at least one additional biomarker in the biological sample, and a reference standard indicating a reference concentration of the at least one additional biomarker of cancer in the biological sample.

33: The kit of claim 31, wherein the at least one reagent comprises at least one antibody capable of specifically binding laminin gamma-2 monomer.

34: The method of any of claims 1-11, wherein the cancer is selected from colorectal cancer and bladder cancer.

35: A method for providing a diagnosis of cancer to a subject, the method comprising the steps of:
a. obtaining a biological sample comprising blood from the subject;
b. submitting the blood sample for determination of the concentration of laminin gamma-2 monomer in the biological sample from the subject; and
c. providing a diagnosis to the subject of whether the subject has or does not have cancer wherein said diagnosis is obtained by comparing the laminin gamma-2 monomer concentration from the sample determined in step b) to a reference laminin gamma-2 monomer concentration value, wherein the subject is provided a diagnosis of having cancer if a laminin gamma-2 monomer concentration in the sample is greater than the reference laminin gamma-2 monomer concentration value and the subject is provided a diagnosis of not having cancer if a laminin gamma-2 monomer concentration in the sample is less than the reference laminin gamma-2 monomer concentration, and instructing the subject to seek further medical treatment if the subject is provided with a diagnosis of cancer.

36: A method for diagnosing cancer in a subject, the method comprising the steps of:
a. obtaining a biological sample comprising blood from the subject;
b. submitting the blood sample for determination of the concentration of laminin gamma-2 monomer in the biological sample from the subject;
c. diagnosing the subject as having cancer or not having cancer by comparing the laminin gamma-2 monomer concentration determined in step b) from the sample to a reference laminin gamma-2 monomer concentration value, wherein the subject is diagnosed as having cancer if a laminin gamma-2 monomer concentration in the sample greater than the reference laminin gamma-2 monomer concentration value and the subject is diagnosed as not having cancer if a laminin gamma-2 monomer concentration in the sample is less than the reference laminin gamma-2 monomer concentration

37: A method for providing a risk classification to a subject who is at risk of having cancer, the method comprising the steps of:
a. obtaining a biological sample comprising blood from the subject;
b. submitting the blood sample for determination of the concentration of laminin gamma-2 monomer in the biological sample from the subject; and
c. providing a risk classification to the subject by determining whether or not the subject is at risk or is not at risk for cancer by comparing the laminin gamma-2 monomer concentration determined in step b) from the sample to a reference laminin gamma-2 monomer concentration value, wherein the subject is determined to be at risk for cancer if a laminin gamma-2 monomer concentration in the sample greater than the reference laminin gamma-2 monomer concentration value and the subject is determined as not being at risk for cancer if a laminin gamma-2 monomer concentration in the sample is less than the reference laminin gamma-2 monomer concentration.

38: A method for determining whether a subject is at risk of having cancer, the method comprising the steps of:
a. obtaining a biological sample comprising blood from the subject;
b. submitting the blood sample for determination of the concentration of laminin gamma-2 monomer in the biological sample from the subject; and
c. determining whether the subject as being at risk or not being at risk for cancer by comparing the laminin gamma-2 monomer concentration determined in step b) from the sample to a reference laminin gamma-2 monomer concentration value, wherein the subject is determined to be at risk for cancer if a laminin gamma-2 monomer concentration in the sample greater than the reference laminin gamma-2 monomer concentration value and the subject is determined as not being at risk for cancer if a laminin gamma-2 monomer concentration in the sample is less than the reference laminin gamma-2 monomer concentration.

39: The method of any of claims 35-38, wherein the cancer is selected from digestive cancer and urological cancer.

40: The method of claim 39, wherein the digestive cancer is colorectal cancer and the urological cancer is bladder cancer.

41: The method of any of claims 1-11, wherein the method is performed by an immunoassay which employs monoclonal antibody 2H2.

42. (canceled)

43: A method for determining the concentration of laminin gamma-2 monomer in a blood sample from a subject, the method comprising the steps of obtaining a blood sample from a subject and determining the concentration of laminin gamma-2 monomer in the blood sample.

44: A method of claim 43, wherein the method is performed by an immunoassay.

45: A method of claim 44, wherein the immunoassay employs an antibody and the antibody is monoclonal antibody 2H2.
